(19) United States
(12) Patent Application Publication
(10) Pub. No.: US 2021/0330762 A1
(43) Pub. Date: Oct. 28, 2021

(54) MEDICAL APPLICATION OF MUTANT PLASMINOGEN AND PLASMIN POLYPEPTIDE THERAPEUTICS

(71) Applicant: SHENZHEN BAY LABORATORY, SHENZHEN (CN)

(72) Inventors: Xinli LIN, SHENZHEN (CN); Jiali GAO, SHENZHEN (CN)

(21) Appl. No.: 16/856,107

(22) Filed: Apr. 23, 2020

(51) Int. Cl. A61K 38/48; G01N 33/68

(52) U.S. Cl. CPC A61K 38/484; G01N 33/6803; C12Y 304/21007

(57) ABSTRACT

Provided are methods for producing biologically active mutant recombinant plasminogen polypeptides with desired pharmaceutical properties. The refolded polypeptides may be treated with a plasminogen activator, such as tPA, urokinase, or streptokinase to generate biologically active mutant plasmin polypeptide for pharmaceutical use. Methods are also provided to producing biologically active fusion recombinant mutant plasminogen/plasmin polypeptides that can cross the BBB through receptor mediated transcytosis. The fusion partner may carry the mutant plasminogen/plasmin polypeptides into the brain for increased therapeutic efficiency.

### FIELD OF THE DISCLOSURE

This disclosure relates to methods for selecting and producing mutant plasminogen/plasmin and des-kringle plasminogen/plasmin polypeptides that are both biologically active and resisting α2-antiplasmin inhibition for preventing and treating pathogenic polypeptide caused diseases such as thromboembolism related diseases, Alzheimer's disease, and lung fibrosis.

### BACKGROUND OF THE DISCLOSURE

Dissolution (Fibrinolysis) of a Blood Clot (Thrombus). Breakdown of a fibrin clot into soluble components depends on plasmin (Plm), a serine protease that is derived from the freely circulating zymogenic proenzyme plasminogen (FIGS. 1(A), 1(B), 1(C1)-1(C3) and SEQ ID NO:1). Plasminogen (Plg) binds to both fibrin and fibrinogen, thereby being incorporated into a clot as it is formed. Both urokinase (uPA) and tissue plasminogen activator (tPA) are exquisitely specific serine proteases which convert Plg to Plm. The Plm digests the fibrin in the clot to soluble components to which neither fibrin nor fibrinogen bind. Thus the primary biological function of Plm is to dissolve fibrin deposit either in blood clots or in fibrosis such as lung fibrosis. Other important biological functions include the clearance of pathogenic polypeptide such as the β-Amyloid peptide causing Alzheimer's Disease (AD).

Other parts of the blood's intricate fibrinolytic network include the plasminogen activator inhibitor (PAI), which binds to and inhibit the proteolytic activity of tPA and uPA, and α₂-antiplasmin (α₂-AP), which binds to and inhibit the proteolytic activity of Plm. This disclosure is the concept, practice, and application of selecting and making recombinant mutant Plg/Plm and des-kringle Plg/Plm based therapeutics that not only can efficiently and specifically catalyze polypeptide substrate cleavage but also resist α₂-AP inhibition. The selected mutants can be used for treating diseases such as thromboembolism related illness, Alzheimer's disease (AD), and lung fibrosis.

This disclosure also includes the concept, practice, and application of selecting and making other modified forms of recombinant mutant Plg/Plm and des-kringle Plg/Plm based therapeutic constructs that not only can efficiently and specifically catalyze polypeptide substrate cleavage but also resist α₂-AP inhibition. The modification includes polyethylene glycol modification (PEGylation) for extending the in vivo half-life of the therapeutic protein. The PEGylated native or mutated constructs may not only extend the in vivo half-life but can also reduce or eliminate immunogenicity of the resulting therapeutic agents. The selected PEG-mutant constructs may be used for treating diseases such as thromboembolism related illness, AD, and lung fibrosis.

Another example of the modification is site-specific albumination with human serum albumin. The albuminated-mutant constructs may also extend the in vivo half-life and reduce or eliminate immunogenicity of the resulting therapeutic agents. Specifically, the albumination can be a direct Plg/Plm and des-kringle Plg/Plm fusion, or a fatty acid modification such as palmitic acid modification through a mutant Cys-Plg/Plm and des-kringle Plg/Plm construct, and the resulting albumin binding of the modified construct when delivered in vivo. The selected albuminated-mutant constructs may also be used for treating diseases such as thromboembolism related illness, AD, and lung fibrosis.

This disclosure also includes the concept, practice, and application of selecting and making modified forms of recombinant mutant Plg/Plm and des-kringle Plg/Plm based therapeutic constructs that have different substrate specificity than the wild type enzyme. For example, specific forms may be selected with decreased catalytic efficiency toward fibrin substrate, but remain catalytic active toward small peptide substrates such as β-amyloid peptides that are toxic and capable of killing neurons and causing AD. The selected forms will be more specific toward treating a particular disease with reduced or diminished side effects, such as bleeding side effects. Conversely, specific forms may be selected with increased catalytic efficiency toward fibrin substrate, but decreased activity toward small peptide substrates for specific medical applications such as thrombolytic applications with decreased side effects.

This disclosure also includes the concept, practice, and application of selecting and making fusion recombinant mutant Plg/Plm and des-kringle Plg/Plm based therapeutic constructs that can be used for specific delivery to tissues such as the brain (the central nerve system, CNS), in which specific tags are installed on either the N-terminal or C-terminal of the mutant polypeptide constructs for receptor mediated transcytosis in order to overcome the Blood-Brain Barrier (BBB). The selected fusion constructs may be used for treating neurological diseases such as AD.

Structure of Plasmin and its Des-kringle Derivatives. Plg (FIGS. 1(A), 1(B), 1(C1)-1(C3) and SEQ ID NO:1) is a 791 amino acid single-chain glycoprotein which circulates inertly in the blood but can still bind to fibrin at newly formed blood clots; its activation is effected by digestion of the peptide bond between R⁵⁶¹ and V⁵⁶² by tPA trapped in the blood clot; the newly formed Plm then actively digests the fibrin in the clot, thereby dissolving it (FIG. 1(B)). The activated Plg is transformed into 2 separate subunits interconnected by 2 disulfide bridges. The A chain of the Plm molecule consists of 5 triple-loop disulfide kringle (Kr) domains (approximately 78-80 amino acids each), while the B chain contains a “linker” region of about 20 amino acids and a serine protease domain (approximately 228 amino acids, see FIG. 1(A)).

Through laboratory manipulations, 2 des-kringle variants of Plg with potential pharmacological applications, miniplasminogen (mPlg, SEQ ID NO:2) and microplasminogen (μPlg, SEQ ID NO:3), have been created. mPlg consists of only Kr5, the linker, and the serine protease domain, while μPlg consists of only the linker and serine protease domain itself. Initially mPlg is produced by digestion with neutrophil elastase, while μPlg was produced by pH 11 base-mediated cleavage. mPlg and μPlg are also activated to miniplasmin (mPlm) or microplasmin (μPlm) respectively, by digestion at the peptide bond between R⁵⁶¹ and V⁵⁶² by tPA, uPA, or other plasminogen activators (PAs), again forming 2 separate subunits interconnected by the same 2 disulfide bridges. Removal of either one or both of these disulfide bridges by mutagenesis renders the activated serine protease domain non-functional.

In the serum, the activity of Plm is neutralized by its principle inactivator, α2-AP (SEQ ID NO:4), and has a plasma half-life of only 0.2 seconds. α2-AP binds to specific lysine residues located in Kr5 and other kringle domains first and then the catalytic domain. α2-AP also binds to μPlm, which has relatively longer plasma half-life than plasmin—about 4 seconds, which in many cases is still too short for developing an efficient therapeutic drug to cleave and inactivate pathogenic protein or peptide substrates in serum or tissues.

Plasminogen and plasmin deficiency. Plg deficiency is a serious medical problem; however, currently there are no available plg or plm replacement therapeutics. For example, during thrombolytic therapy with high doses of tPA, uPA, or streptokinase, there is a depletion of Plg that may terminate the efficiency of the thrombolytic drugs. These conditions may be reversed with application of the long serum half life mutant μPlg or μPlm generated from this disclosure. In fact, it has been shown in an animal model that the administration of Plg has restored the thrombolytic potential. In addition, decreased levels of Plg or Plm have been shown in several clinical conditions, including disseminate intravascular coagulation, sepsis, leukemia, hyaline membrane disease, liver disease, lung fibrosis and importantly, Alzheimer's disease. Some of these conditions may benefit from the mutant plasminogen or plasmin polypeptide therapeutics generated from this disclosure. A native plasminogen or plasmin polypeptide therapeutics may not be efficient enough in treating these diseases because of the short serum half life resulting from immediate inhibition in the serum by α2-AP, and other limitations of the native protein.

Example 1: Potential Use of mutant plasminogen and plasmin uolvueutide in Vascular Diseases. One of the potential applications of mutant plasminogen and plasmin polypeptide generated from this disclosure is for peripheral arterial occlusion (PAO), either alone or in a combination with PAs. PAO occurs when a clot blocks artery blood flow to a distant part of the body such as the legs, arms, feet, or hands. A classical early hallmark of PAO is “intermittent claudication” or leg pain during sustained activity which subsides after rest. Continued restriction of blood flow ultimately leads to constant leg or limb pain even at rest, ulcers, tissue death, gangrene, and ultimately may require limb amputation. PAO is really the result of peripheral arterial disease (PAD), in which atherosclerotic plaque build-up on the artery walls leads to obstructed blood flow, leading to ischemia in blood starved limbs of the body.

Current treatments of PAO include angioplasty, stents, and thrombolytic intervention with Activase® or Abbokinase® (uPA). Thrombolytics are not currently approved by the FDA for PAO because they require infusions that last a day or more and are associated with high risk of serious bleeding including stroke.

Treatment of acute myocardial infarction (AMI) is fundamentally quite different from PAO. For one thing, the PAs do not dissolve blood clots themselves, but generate active Plm from Plg to do so. While this may be effective in dissolution of a thrombus in a small myocardial artery, difficulties arise in dissolution of much larger thrombi in peripheral arteries and veins because the clots are long and retracted. Circulation is poor near these clots so the supply of Plg substrate is insufficient; consequently, systemically delivered PAs will not only have difficulty infiltrating the clot, but there will also be insufficient Plg substrate to enable efficient dissolution of the clot. On the other hand, a mutant plasmin polypeptide with longer half serum life will be efficiently dissolving clot by itself, avoiding the Plg substrate depletion problems facing the treatment with PAs.

Thrombolytic intervention against PAO has advanced concurrently with significant technical advances in catheter design and delivery, permitting local drug delivery directly into the clot. However, even under these circumstances, PA mediated clot dissolution can be slow, cumbersome, and only partially effective, requiring 1-2 days. Local drug delivery makes Plm or its des-kringle derivatives attractive alternative candidates for PAO treatment, and a mutant plasmin polypeptide that resists α2-AP inhibition and have longer half life may be more efficient than its native counterpart. In addition, the selected mutant plasmin polypeptide may expand the application of plasmin-based thrombolytic therapy to other blood clotting caused diseases, such as deep vein thrombosis (DVT), acute pulmonary embolism (APE), and acute ischemic stroke. An optimal mutant may be selected using methods described in the disclosure for each medical indication.

Example 2: Potential Use of mutant plasmin polypeptide in Alzheimer's Disease (AD). Alzheimer's disease (AD) was first described by German psychiatrist Alois Alzheimer in 1901. AD is mainly characterized by extracellular plaques and intracellular neurofibrillary tangles. The extracellular plaques are primarily composed of β-amyloid (Aβ) peptides, and the intracellular neurofibrillary tangles are composed of the cytoskeletal protein tau. Aβ is a mixture of peptide from 38 to 43 residues, which is generated from β-amyloid precursor protein (APP) by the action of two proteases, β-secretase (BACE-1) and γ-secretase. Results from the last 20 years of research have supported the amyloid cascade hypothesis. This hypothesis proposes that the overproduction of Aβ peptides (mostly from genetic defect), or the failure to effectively clear this peptide (most of the sporadic AD cases), leads to AD through Aβ toxicity and amyloid deposition, which is also thought to be involved in the formation of neurofibrillary tangles. As a result, therapeutic research toward treatment of AD has mainly aimed at blocking production, hindering aggregation, or enhancing clearance of Aβ peptides. One of the earliest Aβ-based therapeutic applications was immunotherapy using Aβ peptide as a vaccine, although clinical toxicity has prevented further development of this strategy. Using antibodies to Aβ peptides as therapeutic agents has also been conducted in many clinical trials. Since the discovery of β-secretase as a critical enzyme in Aβ formation, the β-secretase inhibitor drug studies have become a major field in AD drug development. The field of γ-secretase inhibitor or modulator has also been an active area of AD drug development. More importantly, the realization of the importance of in vivo Aβ cleavage has opened a new field of developing clearance-based therapeutic applications. FIG. 2 shows the biological formation and clearance of Aβ, and the therapeutic strategies toward the treatment of AD based on the underlying biology.

In normal physiological conditions, the production of Aβ is counterbalanced by its elimination via multiple interrelated processes acting in concert, including proteolytic degradation, cell-mediated clearance, active and passive transport out of the brain, as well as deposition into insoluble aggregates. Although each of these processes contributes to Aβ catabolism, research results emerged in the last 10 years have shown that proteolytic degradation is a particularly important regulator of cerebral Aβ levels and, by extension, AD pathogenesis. Saido and colleagues were the first to examine Aβ degradation in the living animal. Subsequent works have identified many different kind of proteolytic enzymes involved in Aβ catabolism, including zinc-metalloproteases, Cysteine proteases, and serine proteases (for review, see). All of these enzymes have potential therapeutic value for treating AD. However, of all the proteases that are directly involved in degrading Aβ in vivo, only a recombinant form of plasmin (catalytic domain of plasmin, μPlm) has been extensively studied as a potential therapeutic drug. Therefore, presently, developing a plasmin or des-kringle plasmin-based therapeutics is the most practical choice among the Aβ degradation enzymes. In the following description, the terms plasmin polypeptides and plasmin or des-kringle plasmin are used interchangeable. The term des-kringle plasmin is defined as plasmin lacking one or more kringle structures, in whatever the order. In particular, plasmin (Plm), des-kringle Plasmin, and splasmin (μPlm) are used interchangeable. For the same reason, the terms plasminogen polypeptides and plasminogen or des-kringle plasminogen are used interchangeable. Likewise, the term des-kringle plasminogen is defined as plasminogen lacking one or more kringle structures, in whatever the order. In particular, plasminogen (Plg), des-kringle Plasminogen, and μplasminogen (μPlg) are used interchangeable.

All three functional proteases involved in plasmin-based thrombolysis are implicated in Aβ degradation: Plm, tissue-type plasminogen activator (WA), and urokinase-type plasminogen activator (uPA). Of these, only Plm has been shown to directly degrade Aβ in both monomeric and fibrillar forms. tPA and uPA, however, are believed to execute their Aβ-cleavage function through Plg activation and the resulting action of Plm, the same as in thrombolysis in vivo. Since the major therapeutic target in this disclosure is soluble Aβ in the serum, theoretically tPA and uPA may not be able to serve as therapeutic agents here because they can not directly hydrolyze Aβ. In addition, since serum activated Plm is immediately inhibited by α2-AP, tPA and uPA will also not be effective as an indirect Aβ cleaving proteases through plasminogen activation.

Studies in cultured cells have shown that purified Plm significantly decreases the level of neuronal injuries induced by aggregated Aβ. In separate research, Ledesma et al have not only shown that Plm degrades Aβ, converting it from amyloidogenic form to non-amyloidogenic form, but have also shown consistently that the level of Plm is reduced in brain tissues from AD patients.

Despite all of the scientific researches, there have been no published data of using plasmin polypeptides as therapeutic agents to treat AD, therefore the present disclosure represents the first plasmin based therapeutic methods for treating AD.

Another key issue need to be addressed in AD is the difficulties for macromolecules to cross the Blood Brain Barrier (BBB), which has hindered the development of neurotherapeutic agents, especially large molecule drugs for CNS diseases. To facilitate the BBB crossing, this disclosure designed fusion proteins connecting μPlm or plasmin polypeptide to peptides or single-chain Mab (scMab) that function as a receptor-mediated transcytosis carrier. On the other hand, it has been shown that Aβ peptides cross the BBB in AD patients. Research has shown that soluble, blood-born Aβ peptides can cross a defective BBB and interact with neurons in the brain. Furthermore, it has been shown that Aβ peptides may across the intact BBB through the low density lipoprotein receptor related protein 1 (LRP1) mediated cellular uptake. These results suggest that the blood may serve as a major, chronic source of soluble, exogenous Aβ peptides that can cross BBB and bind selectively to certain subtypes of neurons and accumulate within these cells. In addition, published results have shown that peripherally applied Aβ-containing inoculate induced cerebral β-amyloidosis, further implying that clearing peripheral Aβ may be as important as cerebral clearance. A μPlm-based fusion constructs generated from this disclosure may clear both peripheral and cerebral Aβ, further strengthening the therapeutic efficiency.

An optimal mutant plasmin polypeptide may be selected using methods described in the disclosure that can specifically cleave and detoxify the β-Amyloid peptide but have lower catalytic activities toward other common substrates such as fibrin, in addition to resist α2-AP inhibition. The tailor selected mutant plasmin polypeptide drug may therefore have higher efficacy for treating AD but with reduced possible side effects.

Example 3: Potential Use of mutant plasmin polypeptide in lune fibrosis. Reduced fibrinolysis due to plasmin down regulation has been implicated in lung fibrosis. It has shown that mice with targeted deletion of the plasminogen gene have poor outcomes in pulmonary fibrosis conditions. It has also been shown that mice with overexpression of a PAI-1 developed impaired systemic plasminogen activation to plasmin, resulting in a more severe lung fibrotic response following bleomycin injury than do littermate controls, and increased plasminogen activation has anti-fibrotic effects. On the other hand, The plasminogen activation system is impaired in idiopathic pulmonary fibrosis (IPF), further prove that the plasminogen activation system is critical for preventing the IPF disease. Direct evidence of plasmin acting in the lung fibrosis system has also been published.

All these results clearly indicate that a plasmin replacement therapeutics using the selected, high efficient mutant plasmin polypeptide could be an effective therapeutics for lung fibrosis, a devastating disease with no effective treatment currently.

For lung fibrosis treatment, in addition to systemic delivery through intravenous, subcutaneous, or submuscular routes, local delivery using inhale devices directly deliver the mutant plasmin polypeptide into the lung may have advantages for higher therapeutic efficiency and lower side effects. The inhale devices include Nebulizers, metered dose inhalers, and dry powder inhalers. All have advantages and disadvantages in the quest for optimal treatment and convenient use for lung fibrosis. Selection of delivery methods and route of delivery is within the skill of art.

Description of the disclosure. The first crystal structure of μPlm was derived from the E. coli expressed and refolded protein purified from the inventor's lab, see Wang, X.; Lin, X.; Loy, J. A.; Tang, J.; Zhang, X. C., Crystal structure of the catalytic domain of human plasmin complexed with streptokinase. Science 1998, 281 (5383), 1662-5. The structure of the zymogen form of the enzyme, μPlg, was subsequently published using either refolded protein from E. coli (See Wang, X.; Terzyan, S.; Tang, J.; Loy, J. A.; Lin, X.; Zhang, X. C., Human plasminogen catalytic domain undergoes an unusual conformational change upon activation. J Mol Biol 2000, 295 (4), 903-14.) or purified recombinant protein from insect cells. The structure of α2-AP has also been published. Although the complex structure of μPlm and α2-AP has not been published, similar complex of the trypsin-antitrypsin structure may be used as a guide for modeling the surface contact residues for μPlm and α2-AP. This disclosure is to synthesis and select μPlm mutants that not only have similar or better catalytic activity with the wild-type enzyme, but also resist α2-AP inhibition. Structure based design and synthesis of “escape” mutants for proteolytic enzymes has been successful, for example: tPA mutants have been designed and selected from structural information to have similar enzymatic properties as the wild-type enzyme, but displayed significant resistance to inhibition by plasminogen activator inhibitor-1 (PAI-1).

Among the mutagenesis methods, one of the most widely used is alanine-scanning mutagenesis, which is a simple and widely used technique in the determination of the functional role of protein residues. Among the 20 natural amino acids, alanine is the substitution residue of choice because it eliminates the side chain beyond the 0 carbon and yet does not alter the main-chain conformation (as can glycine or proline) nor does it impose extreme electrostatic or steric effects. Using an alanine-scanning method, 112 residues of factor VIIa on the interface of Factor TF/VIIa complex have been examined to determine important contributions of individual residues. Conversely, the method has also been performed on serine protease inhibitor (serpin) to study the interaction with their corresponding serine proteases. The alanine-scanning mutagenesis method may be initially used to screen for mutants that have similar or better activity as the wild-type plasmin polypeptide, but can escape or resist α2-AP inhibition. Selected mutagenesis positions may be further mutated to all other amino acids to identify the best mutant in a particular sequence position. Furthermore, combination of selected mutants at different sequence positions may be constructed and selected to obtain the best therapeutic candidate for a particular medical indication.

The mutants may be refolded from inclusion bodies produced from E. coli. Methods for refolding proteins from E. coli inclusion bodies have been reported. See, e.g., U.S. Pat. No. 6,583,268; U.S. Pub. Nos. US 2003/070242; US 2003/0199676; US 2004/0265298; and US 2005/0227920; and PCT Pub. Nos. WO 03/039491; WO 2004/094344; WO 01/55174; WO 2005/05830; US Pub. Nos. WO/2008/054592; PCT Pub. Nos. PCT/US2007/021061; published article, D. Medynski et al, Protein Expr Purif 52 (2007), no. 2, 395-402. Methods disclosed in these references include steps of refolding proteins by reducing pH slowly from a high pH to a physiological pH, and specifically, for refolding mPlg and μPlg.

The disclosure also provides methods for Cys-PEGylation scanning mutagenesis. As shown in FIG. 3(B), loops 7 and 8 (contact regions between μPlm and the γ-SK) have been identified as potential mutagenesis sites. It has been shown that γ-SK blocks α2-AP inhibition without interfering with μPlm activity, implying that the γ-SK-μPlm complex has the desired properties of being used as a possible drug candidate for AD. However, γ-SK is a bacterial protein and therefore immunogenicity may be an impassable hurdle for Aβ drug development. On the other hand, using polyethylene glycol (PEG) to “replace” the γ-SK domain in the complex may result in similar blockage of α2-AP inhibition and also retain native activity. PEGylated drugs are known to extend in vivo half-life, reduce or eliminate immunogenicity, and have already been approved by the FDA. Two steps are designed for Cys-PEGylation screening at selected residues of loops 7 and 8. First, residues are identified according to preliminary alanine scanning mutagenesis results for Cys mutation and will characterize the kinetic properties of the resulting Cys mutants. Second, for Cys mutants that have desired kinetic properties, Cys-PEGylation will be performed and the PEGylated μPlm (PEG-μPlm) mutants will be characterized. Structurally (see FIG. 3(B)), using polyethylene glycol to functionally “replace” the 7-domain of SK is a novel approach, and the implication of the resulting PEGylated μPlm can be far-reaching in all aspects of drugable properties: native activity, blocking α2-AP inhibition, reducing or eliminating possible immunogenicity, and longer in vivo half-life.

In addition to loops 7 and 8, other positions such as regions in loop 3 or any other loops and positions may also be mutated for Cys-PEGylation selection of desired PEGylated mutants.

The disclosure also provides methods for fusion construct for crossing the blood brain barrier (BBB). In this case, a “molecular Trojan horse” strategy is designed by engineering fusion partners to ferry the therapeutic μPlm across the human BBB. Two different fusion partners with proven receptor mediated BBB transcytosis properties are initially designed in order to select high-efficient transporters for our selected μPlm constructs, as shown in FIG. 10 and SEQ ID NO:5 and SEQ ID NO:6.

In the following description, plasminogen polypeptide includes full-length plasminogen (FIGS. 1(A), 1(B), 1(C1-1C3) and SEQ ID NO:1) and varies forms of des-kringle forms plasminogen including but not limited to mPlg (SEQ ID NO:2) and μPlg (SEQ ID NO:3); likewise, plasmin polypeptide includes full-length plasmin and varies forms of des-kringle forms plasmin including but not limited to mPlm and μPlm. Mutant plasminogen or plasmin polypeptides include any and all mutations in the aforementioned plasminogen or plasmin polypeptide that not only have similar catalytic activity than the wild type enzyme but also resist α2-AP inhibition. In addition, mutant plasminogen or plasmin constructs include all possible fusion and PEGylated and other modified forms of mutant plasminogen or plasmin polypeptides described in the disclosure.

All references, publications, and patent applications disclosed herein are hereby incorporated by reference in their entirety.

### BRIEF SUMMARY OF THE DISCLOSURE

The disclosure provides methods for producing biologically active mutant recombinant plasminogen polypeptides with desired pharmaceutical properties. The refolded polypeptides may be treated with a plasminogen activator, such as tPA, urokinase, or streptokinase to generate biologically active mutant plasmin polypeptide for pharmaceutical use.

The disclosure also provides methods for producing biologically active fusion recombinant mutant plasminogen/plasmin polypeptides that can cross the BBB through receptor mediated transcytosis. The fusion partner may carry the mutant plasminogen/plasmin polypeptides into the brain for increased therapeutic efficiency.

The disclosure also provides methods for producing biologically active PEGylated recombinant mutant plasminogen/plasmin polypeptides. The resulting PEG-plasminogen and PEG-plasmin may have longer in vivo half-life as well as reduced or diminished immunogenicity. The PEGylation position may be specific or non-specific, depending on the requirements.

The disclosure also provides methods for producing other biologically active, modified forms of recombinant mutant plasminogen/plasmin polypeptides such as albuminated forms of recombinant mutant plasminogen/plasmin polypeptides with desired pharmaceutical properties.

The disclosure provides methods for making and selecting the aforementioned mutant, fusion, PEGylated and other modified forms of plasmin polypeptides with desired pharmaceutical properties. These methods are within the skill of art.

Although the foregoing disclosure has been described in some detail by way of illustration for purposes of clarity and understanding, it will be apparent to those skilled in the art that certain changes and modifications may be practiced. Therefore, descriptions and examples should not be construed as limiting the scope of the disclosure, which is delineated by the appended claims.

### BRIEF DESCRIPTION OF THE DRAWINGS

FIG. 1(A) shows a schematic presentation or plasminogen structure, with kringles (K1-K5) and μPlasmin structures labelled.

FIG. 1(B) is a diagram that shows the mechanism of a newly formed Plm actively digests fibrin in a clot, thereby dissolving it.

FIG. 1(C1)-FIG. 1(C3) together form a sequence and shows the native plasminogen cDNA sequence and the protein translation (SEQ ID NO:1) (NM_000301.2; GI:186972151). The nucleotide sequence numbers are labeled at the left, and protein sequence numbers (starting from Glu-Plg) are labeled at the right as superscripts. The protein sequence of recombinant mPlg is labeled underlined (A⁴⁴⁰-N⁷⁹¹), and that of the μPlg is labeled underlined and bold (A⁵⁴²-N⁷⁹¹).

FIG. 2 shows the Aβ formation, clearance, and therapeutic strategies. The right side of the flow chart describes the formation of Aβ from APP by in vivo cleavage of APP with the β-secretase and the γ-secretase, and the biological clearance mechanism of Aβ. The left side of the flow chart describes the therapeutic strategies based on the mechanism.

FIG. 3(A) shows Loop structures and residues around the active cleft of μPlm. The μPlm structure from published literature were used. Numbering system is derived from full length Plg. The structure is viewed with Cn3D software available from the NCBI Structure site. Backbone: Wire without sidechains; Loops: Tubes with protein sidechains labeled with Wire; Active side triad, both backbone and sidechains are labeled with Ball and Stick. A total of 36 amino acids are labeled as the loop structure here. Selected sidechains in the loop structures are labeled in blue letters. Abbreviations for the amino acid residues are as follows: A, Ala; D, Asp; E, Glu; F, Phe; H, His; K, Lys; L, Leu; N, Asn; P, Pro; Q, Gln; R, Arg; S, Ser; T, Thr; V, Val; W, Trp; and Y, Tyr.

FIG. (3B) shows the contact region between μPlm and the γ-domain of SK. The SK-μPlm complex structure from published literature were used. Label: μPlm Backbone: Wire without sidechains; γ-domain of SK backbone: tube wom. Loops: both Backbone and protein sidechains labeled with Ball and Stick; Active side triad, both backbone and sidechains are labeled with Ball and Stick. A total of 18 amino acids are labeled as the loop structure here. Two loops and selected amino acid side chains are labeled.

FIG. 4 shows nucleotide and amino acid sequence of μPlg. Sequence of the synthetic gene is shown here, along with the amino acid sequence. The synthetic gene is optimized for E. coli expression. Sequences of the six loops identified in FIG. 3(A) and two loops identified in FIG. 3(B) are shown.

FIG. 5(A) shows SDS-PAGE analysis of μPlg before activation to μPlm by uPA. Lanes: 1=Mol. Weight Markers, 2=μPlg non-reduced, 3=μPlg reduced.

FIG. 5(B) shows portion of μPlm after activation with uPA. The triangles indicated the 2 digestion sites within μPlg-1, both near the amino terminus of the expressed, refolded product. Underlined site indicates the authentic activation site, boxed site indicates engineered recognition site.

FIG. 5(C) shows Hanes plot kinetic comparison of refolded μPlm and commercially available plasmin (Sigma, St. Louis, Mo.) against chromogenic substrate S-2403 (Chromogenix, Goteberg, Sweden). The kinetic parameters are summarized in the box below the Hanes plot.

FIG. 6(A) shows SDS-PAGE analysis (with β-ME reduction) of whole cell extracts of cells containing expression plasmids for μPlg or mPlg after induction with IPTG for 3 hrs. Lanes: Lane 1. MW std; Lane 2. μPlg −IPTG induction; Lane 3. μPlg +IPTG induction; Lane 4. mPlg −IPTG induction; Lane 5. mPlg +IPTG induction.

FIG. 6(B) shows SDS-PAGE (with β-ME reduction) analysis of purified μPlg and mPlg inclusion bodies. Lanes: Lane 1. MW std; Lane 2. μPlg; Lane 3. mPlg.

FIG. 7(A) shows Superdex 75 chromatogram of refolded μPlg.

FIG. 7(B) shows SDS-PAGE of μPlg fractions isolated in FIG. 7(A). Lanes: Lane 1. MW std; Lane 2. F 42 non reduced; Lane 3. F42 reduced; Lane 4. F50 non-reduced; Lane 5. F50 reduced. Comment: non-reduced, non-activated (full length) μPlg has an apparent mobility faster (Mr˜29 KDa) than the reduced form (Mr˜32 KDa) (lane 4 vs. lane 5). The lower molecular weight bands in the reduced lane 3 represent auto-activated fragments.

FIG. 7(C) shows Superdex 75 of refolded mPlg. The elution peak on the left consists of multimeric unfolded forms of mPlg while the protein peak on the right consists primarily of mPlg monomers.

FIG. 7(D) shows SDS PAGE of Superdex 75 chromatography in FIG. 7(C). Lanes: Lane 1. MW std; Lane 2. non-reduced F28, Lane 3. reduced F28; Lane 4. non-reduced F45; Lane 5. reduced F45.

FIG. 7(E) shows a sample of purified mPlg stored in alkaline pH buffer (20 mM Tris, pH 9.0, 0.2 M arginine, 0.15 M NaCl) auto-activated after storage at 4° C. for 1 week. Lanes: Lane 1. MW std; Lane 2. reduced sample of mPlg.

FIG. 8(A) shows an example of the WT μPlm purification: P1 is the unfolded polymer peak, and P2 is the refolded peak. A non-reduced SDS-PAGE is shown in the insert.

FIG. 8(B) shows the same procedure for the purification of the mutant F587A.

FIG. 8(C) shows SEC purification summary of the WT and 5 mutants with various refolding efficiency.

FIG. 8(D) shows an example of a Michaelis-Menten plot of the WT enzyme, showing the excellent fit of the assay data.

FIG. 8(E) shows a summary table of the kinetic parameters of the wild-t (WT) μPlm and 10 refolded and purified mutants: T581A, R582A, G584A, M585A, F587A, K607A, S608A, P609A, P611A, and Q622A. The WT and F587A are high-lighted for comparison.

FIG. 8(F) shows a plot of catalytic efficiency of the WT and 10 different mutants from E. The WT and F587A are indicated with arrows for comparison.

FIG. 8(G) shows the inhibition curve of WT μPlm at different α2-AP concentration. The reverse triangle at the right side of the figure shows the increased concentration of α2-AP downward. The highest concentration of the α2-AP is two fold of the μPlm, and the next high concentration is one fold.

FIG. 8(H) shows the inhibition curve of F587A. Again, the reverse triangle at the right side of the figure shows the increased concentration of α2-AP downward. The highest concentration of the α2-AP is two fold of the μPlm, and the next high concentration is one fold.

FIG. 8(I) shows a plot of α₂-AP inhibition of the WT μPlm and 10 different mutants. The F587A mutant stands out in the figure as the identified “escape” mutant.

FIG. 8(J) shows a comparison of α₂-AP inhibition of the WT μPlm and the F587A mutant. The figure shows that at the α₂-AP concentration that completely inhibited the μPlm activity, there is still 50% activity remaining for the F587 mutant.

FIG. 8(K) shows a space filling model of the Loop-3 regions of μPlm, indicating the spatial proximity of the active site Serine (S741, mutated to A741 in the crystal structure⁸⁶) and the F587 residue.

FIGS. 9(A)-9(T) show purification of refolded wild type and nineteen F587 mutants. In which. FIG. 9(A) shows the purification of refolded wild type mutant; FIG. 9(B) shows the purification of F587A mutant; FIG. 9(C) shows the purification of F587C mutant; FIG. 9(D) shows the purification of F587D mutant; FIG. 9(E) shows the purification of F587E mutant; FIG. 9(F) shows the purification of F587G mutant; FIG. 9(G) shows the purification of F587H mutant; FIG. 9(H) shows the purification of F587I mutant; FIG. 9(I) shows the purification of F587K mutant; FIG. 9(J) shows the purification of F587L mutant; FIG. 9(K) shows the purification of F587M mutant; FIG. 9(L) shows the purification of F587N mutant: FIG. 9(M) shows the purification of F587P mutant; FIG. 9(N) shows the purification of F-587Q mutant; FIG. 9(O) shows the purification of F587R mutant; FIG. 9(P) shows the purification of F587S mutant, FIG. 9(O) shows the purification of F587T mutant: FIG. 9(R) shows the purification of F587V mutant FIG. 9(S) shows the purification of F587W mutant; FIG. 9(T) shows the purification of F587Y mutant. The Superdex-75 purification profiles of the refolded mutants (each labeled with single letter amino acid) and the non-reduced SDS-PAGE is shown. The refolded peak (second peak in each graph) for each mutant is indicated by an arrow. A summary of the refolding yield is shown in Table 2, in which an integration of the second, refolded peak area is divided by the total peak area and expressed as the percentage yield.

FIG. 10 shows a model of fusion constructs of μPlg for crossing the BBB. Two examples are: (1) Angiopep-2 (AP2) transports to the brain through LRP1 and has been successfully tested for brain delivery. Fusion construct follows, AP2-μPlm (SEQ ID NO:5): MTFFYGGSRGKRNNFKTEEY-SGPGGGS-μPlm. (2) The low-density lipoprotein receptor (LDLR)-binding domain of apolipoprotein B (ApoB) has proven transcytosis property across the BBB and has been successfully used in brain delivery of a therapeutic protein. Fusion construct follows, ApoB-μPlm (SEQ ID NO:6): MSSVIDALQYKLEGTT RLTRK RGLKLATALS LSNKFVEGSHNSTVSPQQQS-μPlm.

### DETAILED DESCRIPTION OF THE DISCLOSURE

The instant disclosure provides methods for the production of mutant recombinant, biologically active plasminogen and plasmin polypeptides. The instant disclosure also provides compositions (including pharmaceutical compositions) comprising biologically active mutant plasminogen and plasmin polypeptides.

The instant disclosure also provides methods for the production of fusion mutant recombinant, biologically active plasminogen and plasmin polypeptides. The instant disclosure also provides compositions (including pharmaceutical compositions) comprising biologically active fusion mutant plasminogen and plasmin polypeptides.

The instant disclosure also provides methods for the production of PEGylated mutant recombinant, biologically active plasminogen and plasmin polypeptides. The instant disclosure also provides compositions (including pharmaceutical compositions) comprising biologically active PEGylated mutant plasminogen and plasmin polypeptides. The PEGylation sites can be primary amine or free cysteine. Specifically, selection methods for specific Cys-Pegylation are described.

The instant disclosure also provides methods for the production of albuminated forms of mutant recombinant, biologically active plasminogen and plasmin polypeptides. The instant disclosure also provides compositions (including pharmaceutical compositions) comprising biologically active albuminated mutant plasminogen and plasmin polypeptides. The albuminated form can be a direct fusion of plasminogen polypeptide with albumin or indirected albumination such as a chemically modified plasminogen polypeptide with a fatty acid such as palmitation. The palmitated polypeptide will then combined with serum albumin in the serum when delivered in vivo through intravenous (IV) route.

The practice of the present disclosure will employ, unless otherwise indicated, conventional techniques of immunology, molecular biology, microbiology, cell biology and recombinant DNA, which are within the skill of the art. See, e.g., Molecular Cloning. a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

It should be noted that, as used herein, the singular form “a”, “an”, and “the” includes plural references unless indicated otherwise; it should also be understood that aspect and embodiments of the disclosure described herein include “consisting” and/or “consisting essentially of” aspects and embodiments.

### Plasminogen and Plasmin Polypeptides

Plasminogen polypeptide includes any naturally occurring species, such as full length protein from any mammalian (e.g., human, farm animals, sport animals, pets, primates, horses, dogs, cats, mice and rats), biologically active polypeptide fragments, and variants (including naturally occurring and non-naturally occurring), including functionally equivalent variants which do not significantly affect their biological properties and variants which have enhanced or decreased activity. Mutant plasminogen polypeptide includes the above plasminogen polypeptides with designed and selected mutations, which are not only having similar biological activity as that of the wild type plasminogen polypeptide but also having increased resistant to α2-AP inhibition.

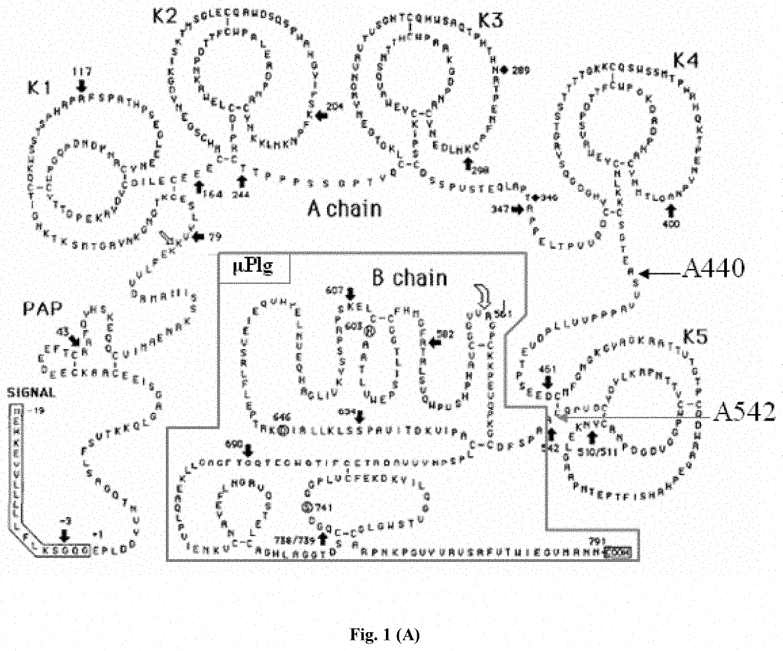

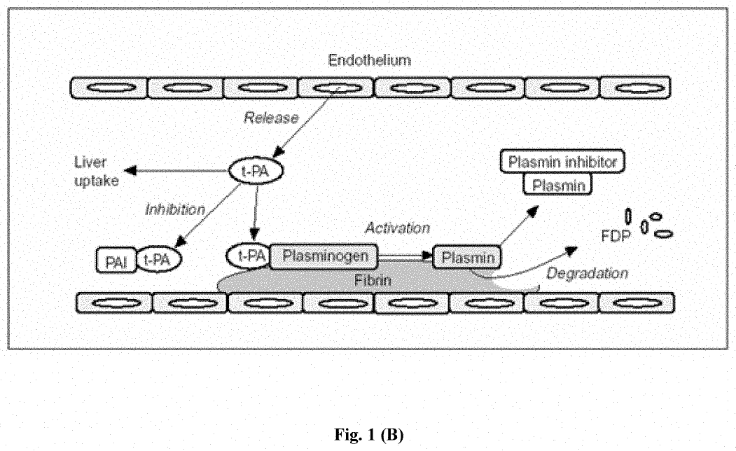

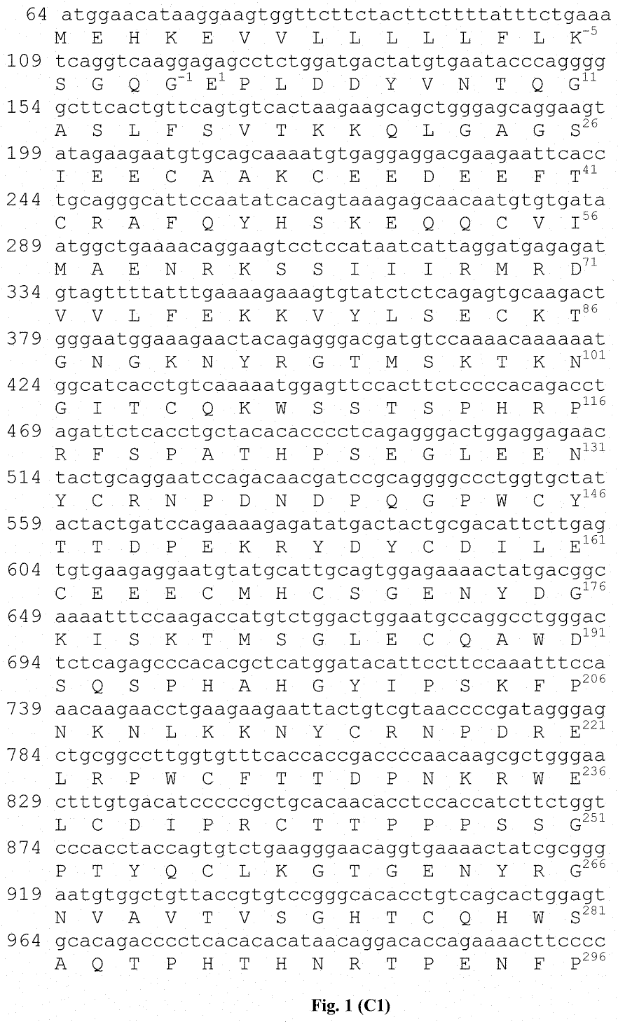

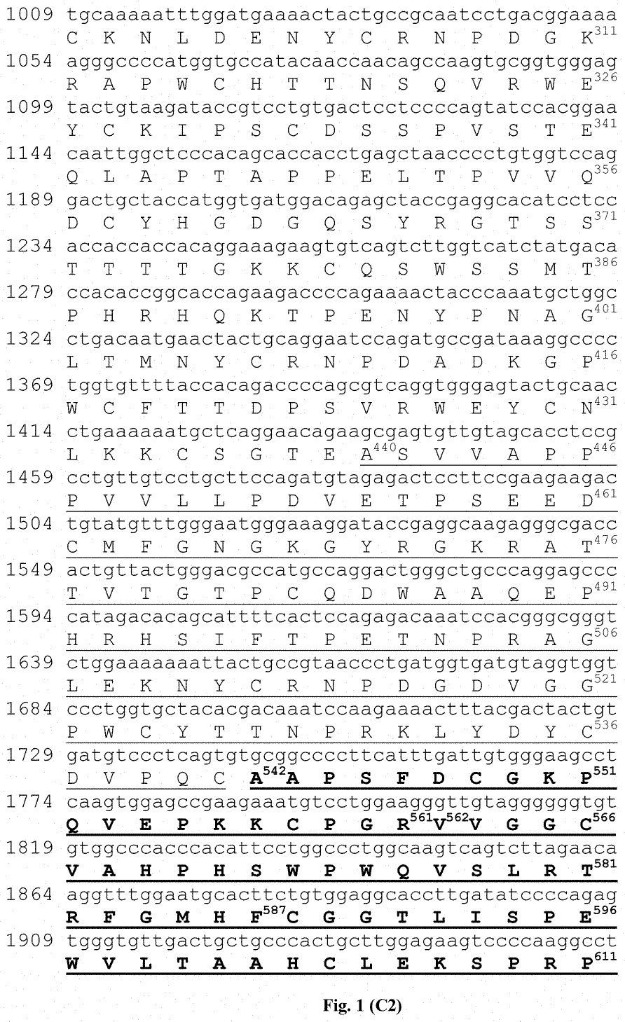

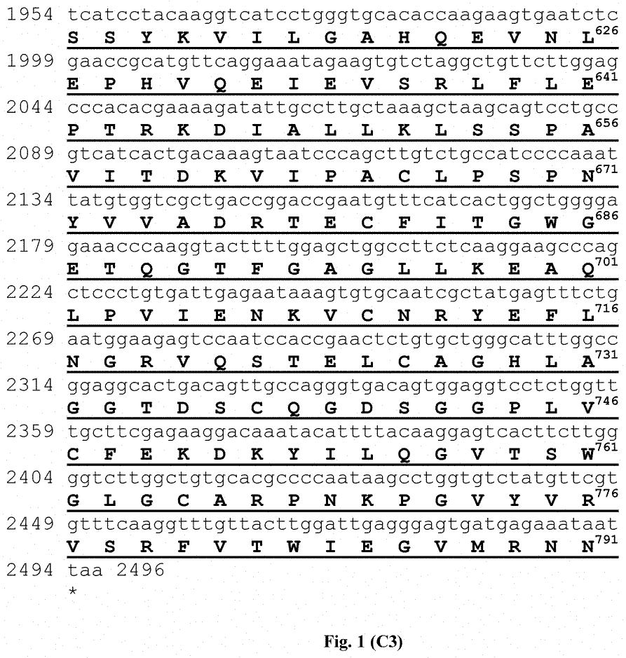

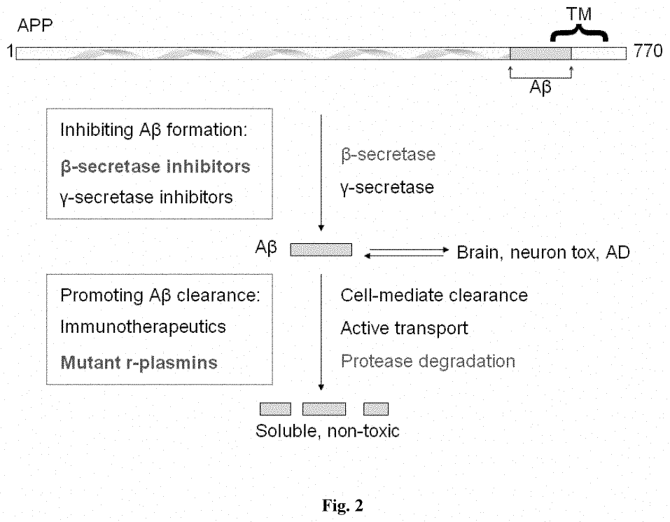

Full length naturally occurring human plasminogen with signal peptide is shown in FIG. 1(C1)-FIG. 1(C3) (protein numbering starts from the first amino acid of the mature Glu-Plg). In some embodiments, the plasminogen polypeptide comprises the amino acid residues 440-791 (mPlg); in some embodiments, the plasminogen polypeptide comprises the amino acid residues 542-791 (μPlg) of protein sequence shown in FIG. 1(A), FIG. 1(B) and FIGS. 1(C1)-C3). In some embodiments, one or more amino acid residues from 440-791 or 542-791 of FIGS. 1(C1)-1(C3) may be deleted.

Variants of the mutant plasminogen polypeptide of the present disclosure may include one or more amino acid substitutions, deletions or additions that do not significantly change the catalytic activity of the enzyme, but are significantly resistant to α2-AP inhibition. To improve or alter the characteristics of the plasminogen polypeptides, protein engineering may be employed. Recombinant DNA technology known to those skilled in the art may be used to create novel mutant proteins or mutants including single or multiple amino acid substitutions, deletions, additions or fusion proteins. Such modified polypeptides can show, e.g., enhanced activity, increased stability, and resistant to α2-AP inhibition. In addition, they may be refolded more efficiently, purified in higher yields, and show better solubility than the corresponding natural polypeptide, at least under certain purification and storage conditions. Thus, plasminogen polypeptide also encompasses derivatives and analogs that have one or more amino acid residues deleted, added, or substituted to generate polypeptides that are better suited for expression, scale up, etc., in the host cells chosen. In some embodiments, amino acid sequences of the desired plasminogen variants are at least about any of 70%, 75%, 80%, 85%, 90%, 95%, 96%, 97%, 98%, or 99% identical to a naturally occurring plasminogen (such as from a human plasminogen).

A mutant plasmin polypeptide is said to have “similar” biological activity if the proteolytic activity (defined as Kcat/Km) of the mutant toward a peptide or protein substrate is about 0.2 time, about 0.4 time, about 0.6 time, about 0.8 time, about 1.0 time, about 1.5 times, about 2.0 times, about 3.0 times, about 4.0 times, about 5.0 times, about 10 times or higher than the wild type plasmin polypeptide.

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

The mutant variants of plasminogen polypeptides also encompass fusion proteins comprising the plasminogen polypeptide. Biologically active plasminogen polypeptides may be fused with sequences, such as sequences that enhance the crossing of the Blood Brain Barrier (BBB), facilitate the coupling of the polypeptide to a support or a carrier, or facilitate refolding and/or purification (e.g., sequences encoding epitopes such as Myc, HA derived from influenza virus hemagglutinin, His-6, FLAG, SUMO, or the His-Tag shown in Table 3 of U.S. Pub. No. 2005/0227920). These sequences may be fused to plasminogen polypeptide at the N-terminal end or at the C-terminal end. In addition, the protein or polynucleotide may be fused to other polypeptides which increase its function, or redirect to another organ in the body, such as redirecting to the brain by facilitating the crossing of BBB, or specify its localization in the cell, such as a secretion sequence. Fusion proteins may also be constructed to increase the in vivo stability of the mutant variants of plasminogen polypeptide, for improved pharmaceutical properties. Fusion proteins may also be constructed to increase the resistance of plasmin polypeptide to α2-AP resistance. Methods for producing recombinant fusion proteins described above are known in the art. The recombinant fusion protein may be produced, refolded and isolated by methods well known in the art.

The mutant variants of plasminogen polypeptides also encompass PEGylated proteins comprising the plasminogen polypeptide. Site-specific PEGylation may be designed by first changing a chosen residue to a Cysteine (Cys), and then specifically PEGylate the Cys residue with various lengths of polyethylene glycols. Methods for producing Cys-PEGylated polypeptides are known in the art. The PEGylated protein may be produced and purified by methods well known in the art.

Mutant plasminogen polypeptides also include functional equivalent variants. Functional equivalent variants are identified and characterized by any (one or more) of the following criteria: after being activated by a plasminogen activator, (a) ability or specifically to digest fibrin; (b) ability or specifically to digest Aβ peptides, (c) ability or specifically to digest other protein or peptide pathogens, (d) ability to resist inhibition by α2-antiplasmin (α2-AP), and (e) ability to digest L-Pyroglutamyl-L-Phenylalanyl-L-Lysine-para-Nitroaniline hydrochloride or other serine protease substrates including natural and synthetic proteins or polypeptides. Biological activity of variants of mutant plasminogen polypeptides may be tested using methods known in the art and methods described herein. In some embodiments, functional equivalent variants have at least about any of 50%, 60%, 70%, 75%, 80%, 85%, 90%, or 95% of activity as compared to full length mutant plasminogen with respect to one or more of the biological assays described above (or known in the art). In some embodiments, functional equivalent variants have the properties of 50%, 60%, 70%, 75%, 80%, 85%, 90%, 95%, 99% or higher than 99% more resistant to inhibition by α2-AP.

The disclosure also provides mutant plasmin polypeptides generated by cleavage of the peptide bond between arginine 561 and valine 562 (amino acid numbering is based on the numbering in FIGS. 1(C1)-1(C3) of mutant plasminogen polypeptides described herein.

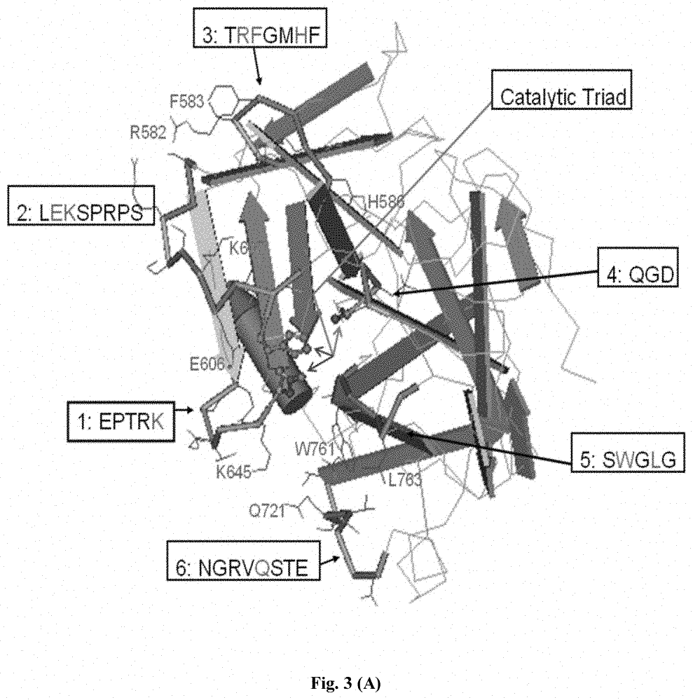

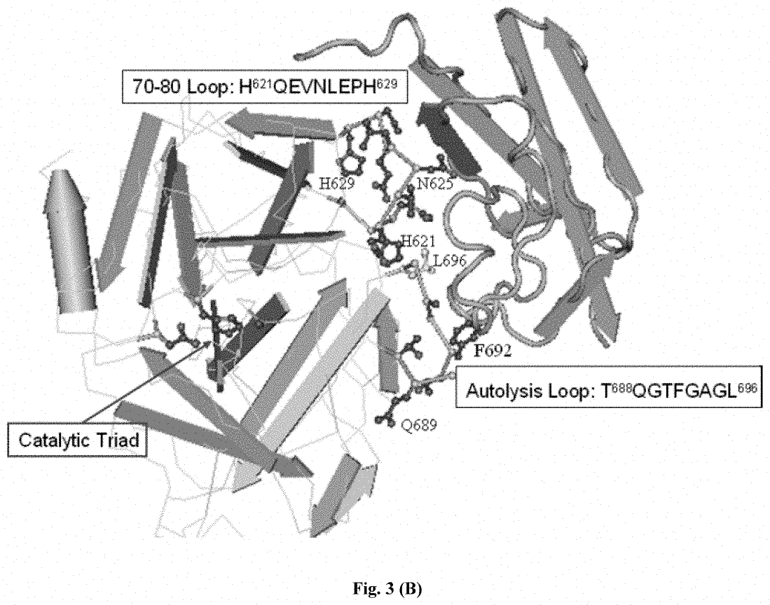

The disclosure also provides methods for selecting residues around the active-site cleft for mutagenesis design. Generally, because serpins inhibit their corresponding proteases by blocking or/and modifying the active site, the binding interface of a protease and its serpin is around the active site on the protease side^{90,91}. The disclosure identified residues around the catalytic cleft and adjacent loops in plasminogen polypeptide that might directly or indirectly involve in the binding of plasmin polypeptide and α2-AP (FIG. 3(A)). Each of these residues is designed to be changed to alanine individually to test the “sensibility” of each residue to α2-AP inhibition. Following are residues and features that are identified for initial alanine-scanning mutagenesis (FIG. 3(A)):
Loop 1: E⁶⁴¹PTRK(D⁶⁴⁶), correspond to part of the “94-shunt” loop of Wang, X. et al. in μPlg connected to active site Asp646 (L7 in references Peisach, E.;-Wang, J.; de los Santos, T.; Reich, E.; Ringe, D., Crystal structure of the proenzyme domain of plasminogen. Biochemistry 1999, 38 (34), 11180-8. and Turner, R. B.; Liu, L.; Sazonova, I. Y.; Reed, G. L., Structural elements that govern the substrate specificity of the clot-dissolving enzyme plasmin. J Biol Chem 2002, 277 (36), 33068-74.108.
   Aoki, N.; Sumi, Y.; Miura, O.; Hirosawa, S., Human alpha 2-plasmin inhibitor. Methods Enzymol 1993, 223, 185-97.).
Loop 2: L⁶⁰⁵EKSPRPS, correspond to part of the “60-loop” of Wang, X. et al. in μPlg extend out of the active site His603 (L5 in references Peisach et al. and Turner, R. B. et al.).
Loop 3: T⁵⁸¹RFGMHF, correspond to the “37-loop” of Wang, X. et al. in μPlg (L3 in references Peisach et al. and Turner, R. B et al.).
Loop 4: Q⁷³⁸GD(S741), correspond to part of the “oxyanion stabilizing loop” of Wang, X. et al. in μPlg connected to the active site Ser⁷⁴¹ (L12 in reference Peisach et al; loop 12 is part of the Si specificity pocket of chymotrypsin and trypsin).
Loop 5: S⁷⁶⁰WGLG, correspond to part of the “Si entry frame” loop of Wang, X. et al. in μPlg (L14 in reference Peisach et al.; loop 14 is part of the left side of the Si-specificity pocket).
Loop 6: N⁷¹⁷GRVQSTE, correspond to part of the “methionine loop” of Wang, X. et al. in μPlg (L11 in reference Peisach et al.).

The instant disclosure also provides methods for selecting residues on the contact region of plasmin polypeptide and the γ-domain of SK (FIG. 3(B)) for mutagenesis. In addition to the loop structures identified in FIG. 3(A), structural analysis by Esmon, C. T.; Mather, T., Switching serine protease specificity. Nat Struct Biol 1998, 5 (11), 933-7.98. Veronese, F. M.; Pasut, G., PEGylation, successful approach to drug delivery. Drug Discov Today 2005, 10 (21), 1451-8. revealed that the 7-domain of streptokinase (SK) in the autolysis contact region forms a major topologic collision if α2-AP is “trying” to bind the SK-μPlm complex. This modeling study was used to explain the insensitivity of SK-Plm to α2-AP inhibition. Thus, besides the loop regions around the active site (FIG. 3(A)), the autolysis loop region may be a major contact region with α2-AP. In addition, the calcium binding loop may also be involved in α2-AP binding directly or indirectly. Docking of plasmin polypeptide into the SK-μPlm active site indicates that the γ-domain of SK involved little structural interaction with substrate binding, implying that mutations in this region not only may disrupt α2-AP binding, but also have minimum interference with substrate binding. The disclosure therefore includes these two regions in designing the alanine-scanning mutagenesis (FIG. 3(B)). Following are residues and features of the two loops in FIG. 3(B) that are identified for alanine-scanning mutagenesis:
Loop 7 (70-80 loop): H⁶²¹QEVNLEPH⁶²⁹, correspond to the “ca binding” loop of μPlg in reference of Wang, X. et al. (L6 in references Peisach et al. and Turner, R. B. et al.).
Loop 8 (Autolysis Loop): T⁶⁸⁸QGTFGAGL⁶⁹⁶, correspond to part of the “autolysis loop” of μPlg in reference of Wang, X. et al. (L9 in reference Peisach et al.).

The instant disclosure also provides methods for selecting residues on any amino acid residues of the plasmin polypeptide that when mutated to any other amino acid residues, resulting not only in a similar biological activity compared with the wild type plasmin polypeptide, but also in a reduced plasmin/α2-AP binding and reduced inhibition of the mutant plasmin by α2-AP.

The instant disclosure also provides methods for selecting mutants that have higher specific activity toward a particular type of polypeptide substrates such as β-amyloid peptide, and reduced activity toward other substrates such as fibrin, and vice versa. The resulting “tailor made” mutant therapeutics may have higher clinical efficacy and lower side effects for treating diseases such as Alzheimer's disease, thrombosis diseases, and lung fibrosis.

Conversely and specifically, the instant disclosure also provides methods for selecting mutants that are more active toward fibrin substrates, and less active toward other biological substrates such as β-amyloid peptide. The mutant therapeutics will be more desirable for thromboembolism or fibrosis related diseases.

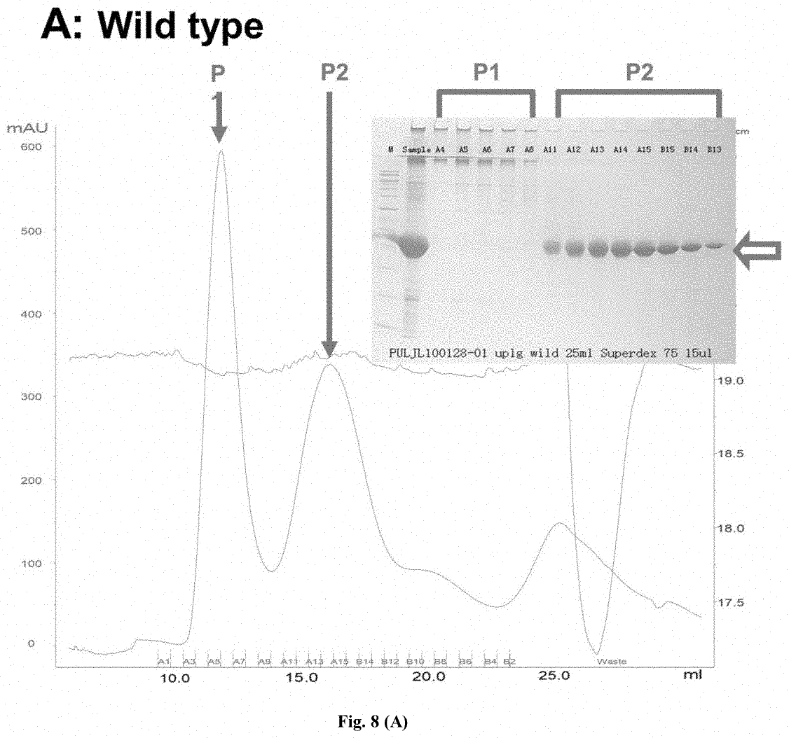

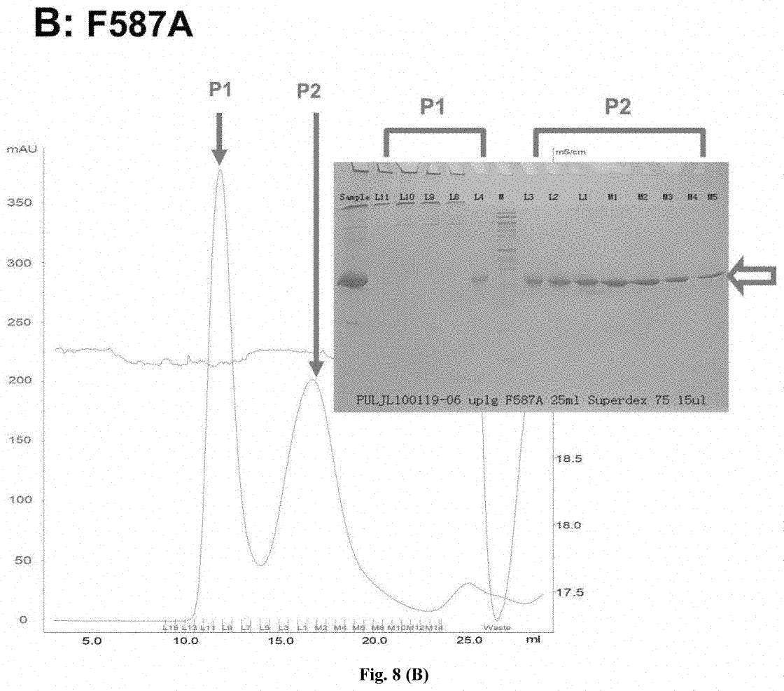

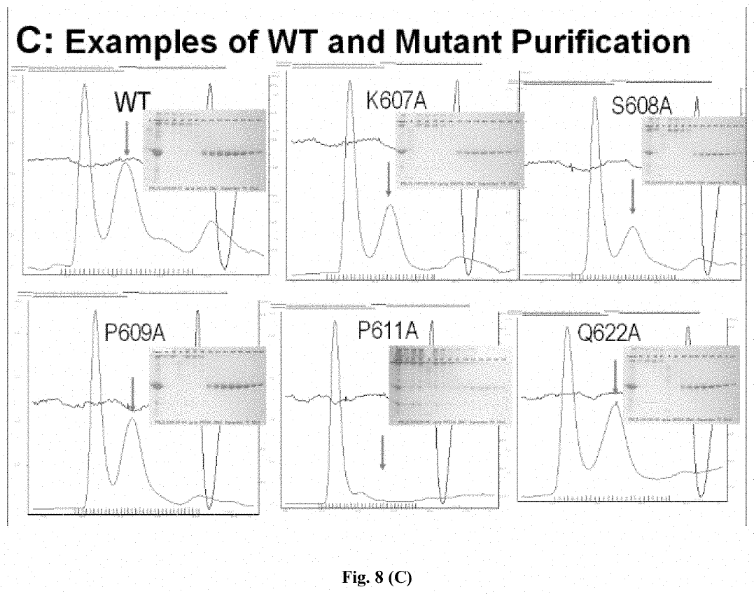

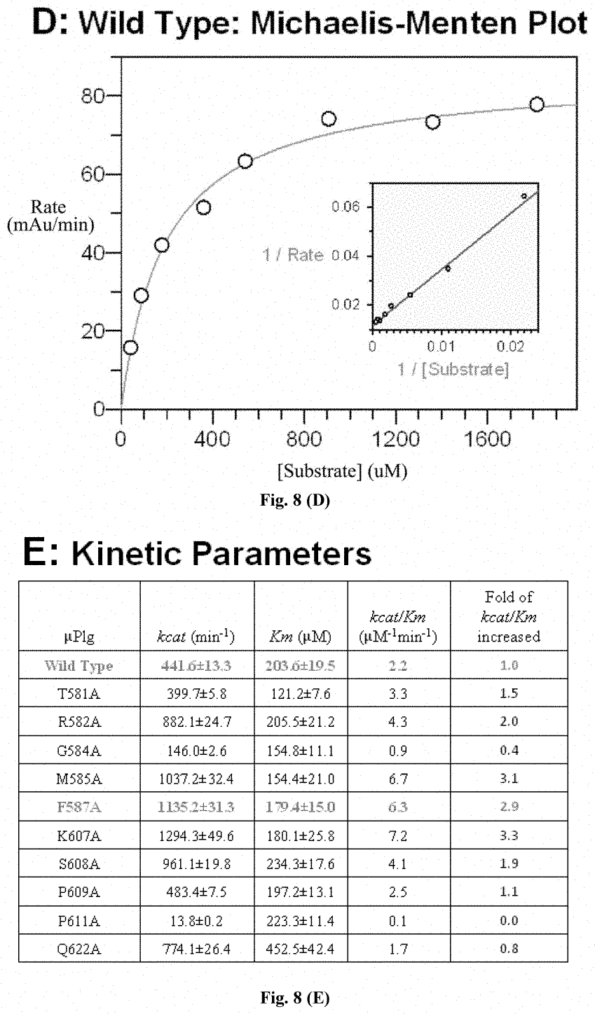

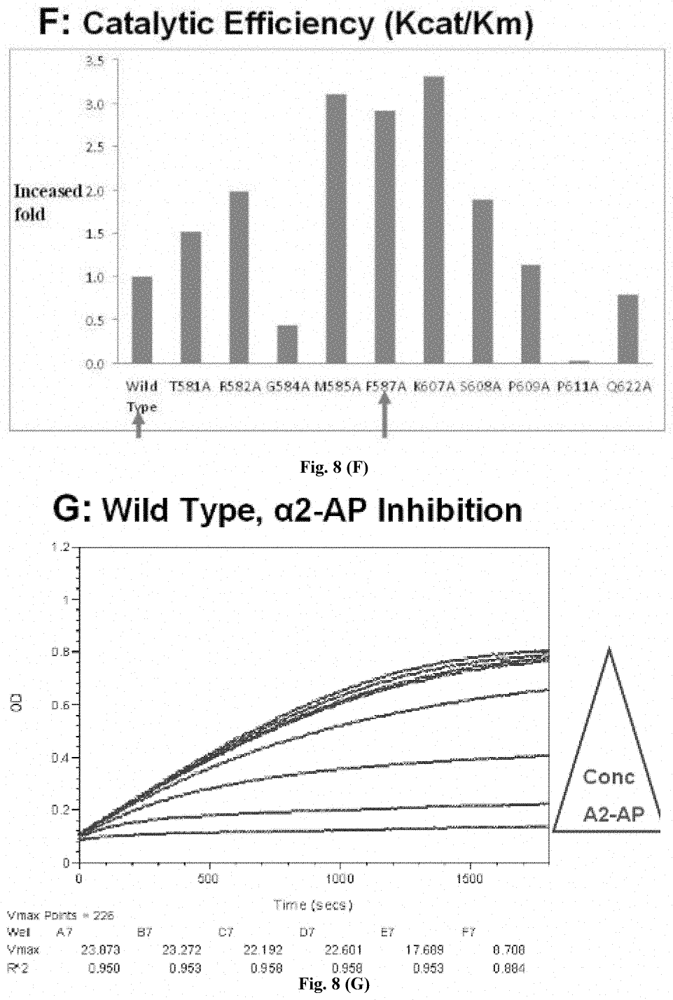

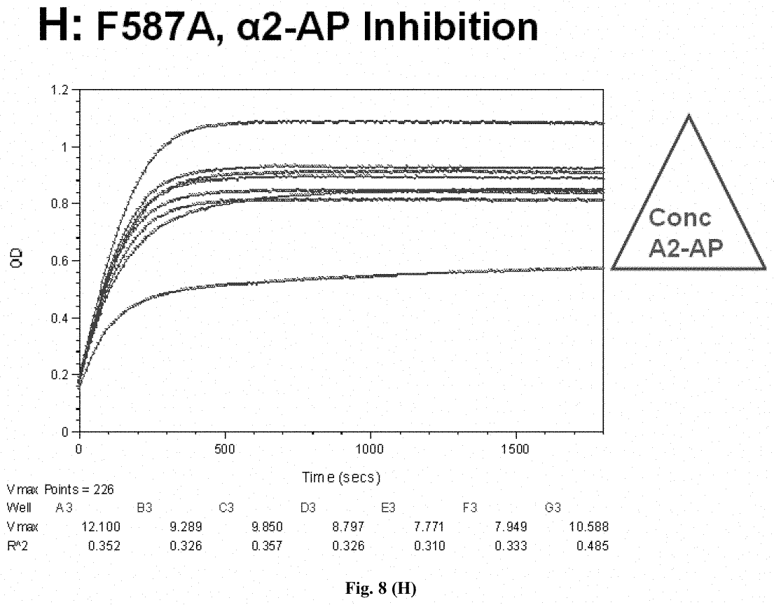

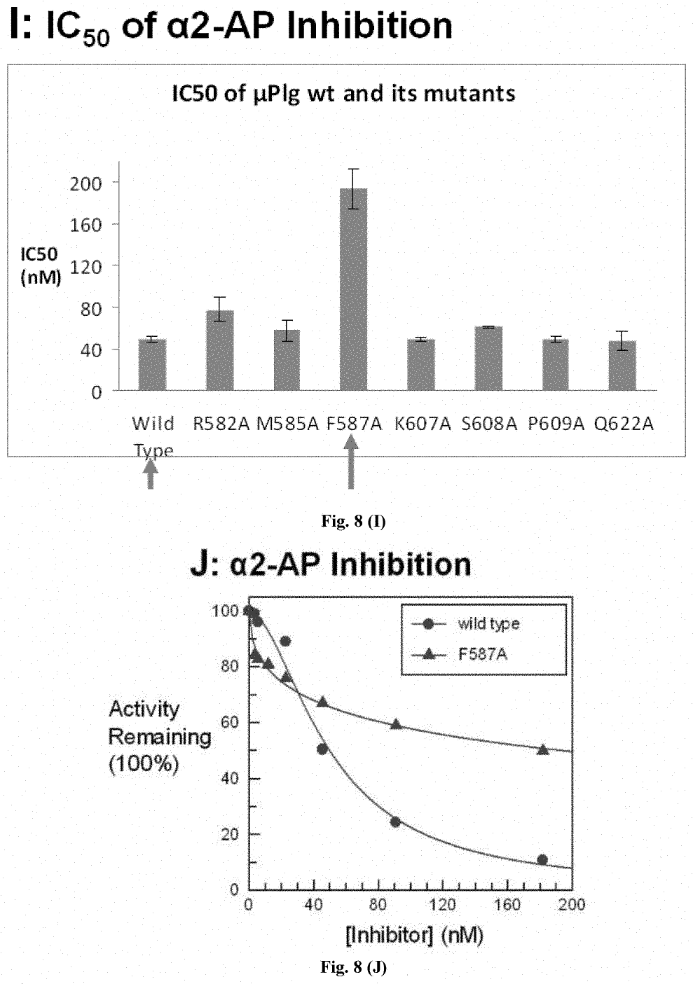

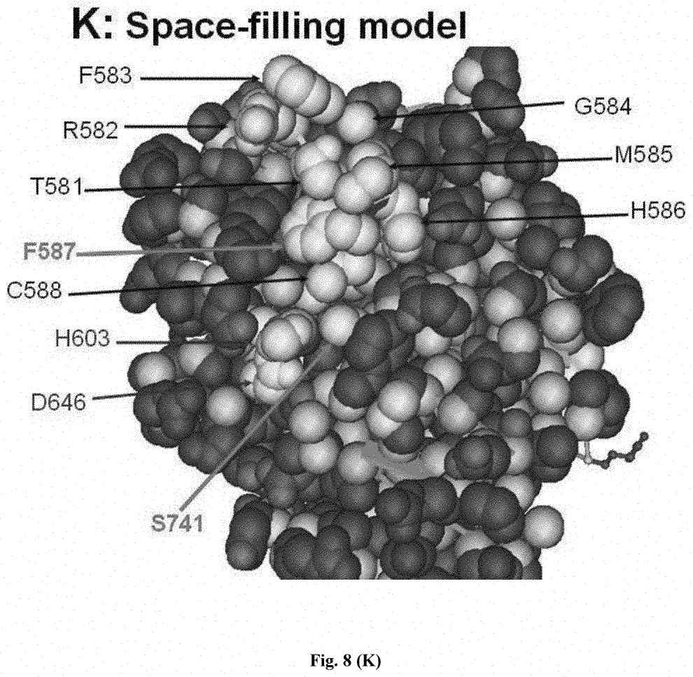

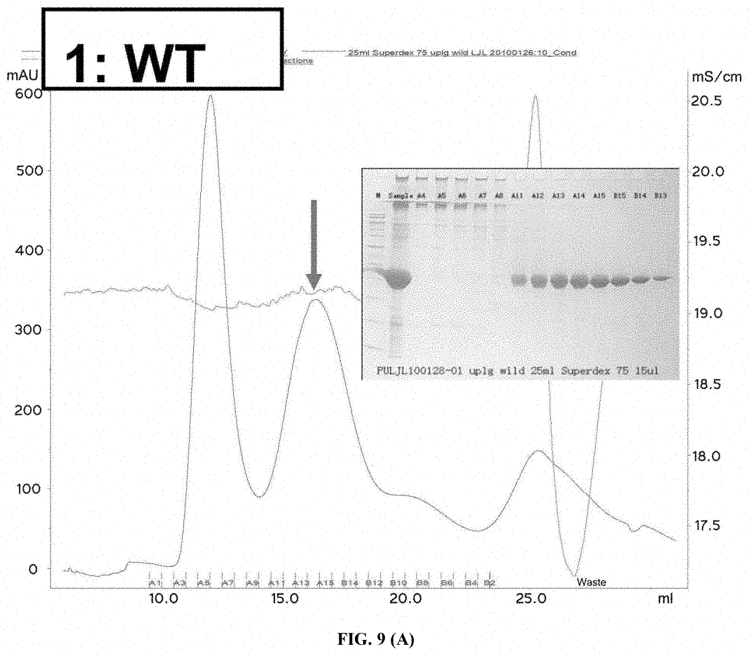

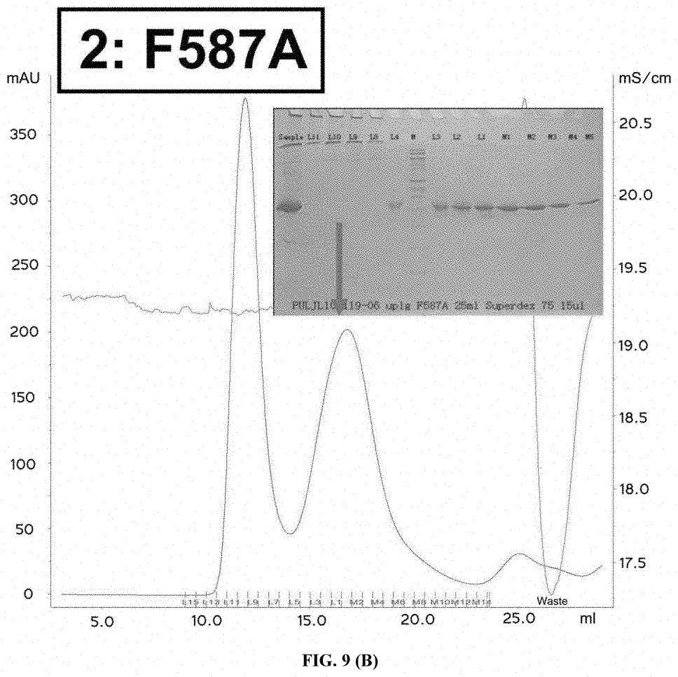

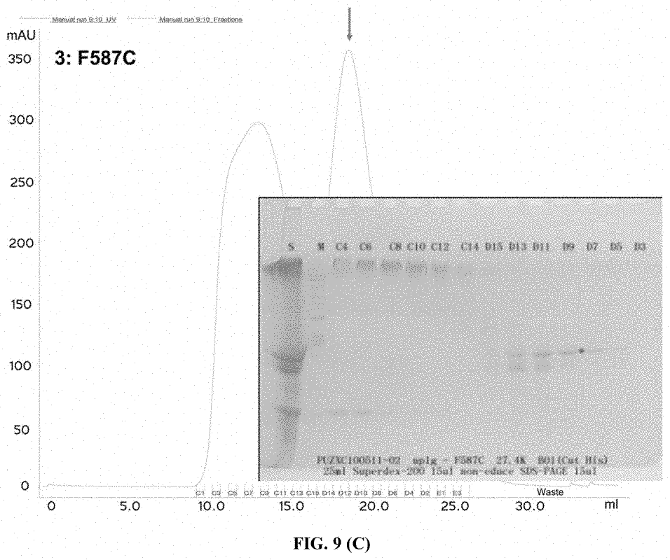

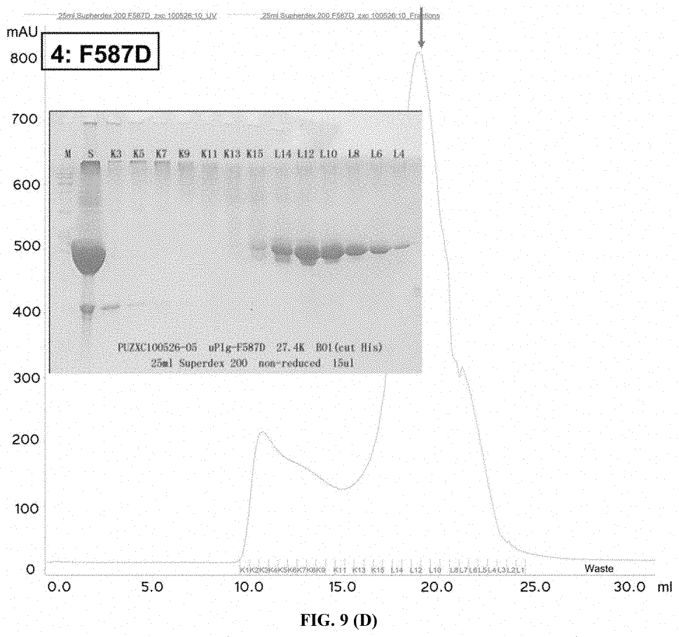

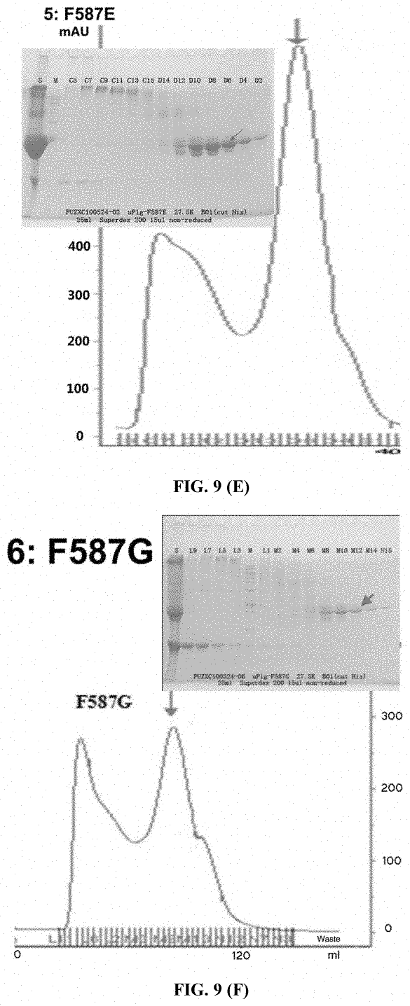

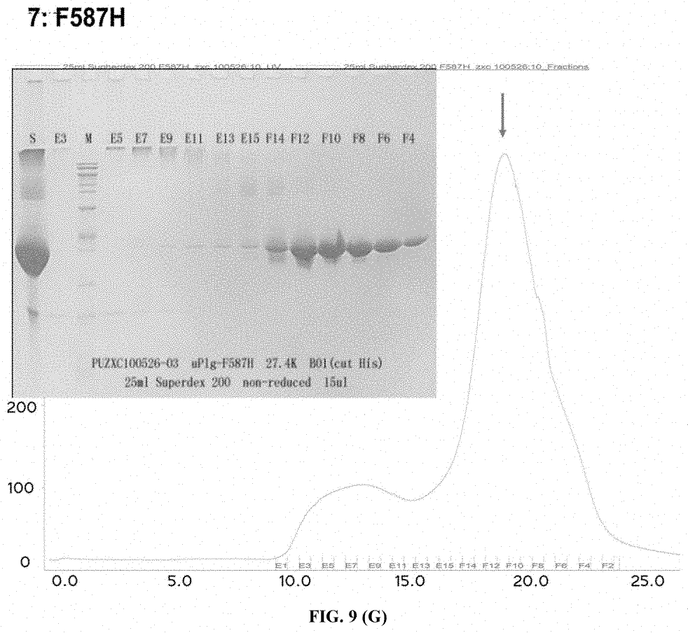

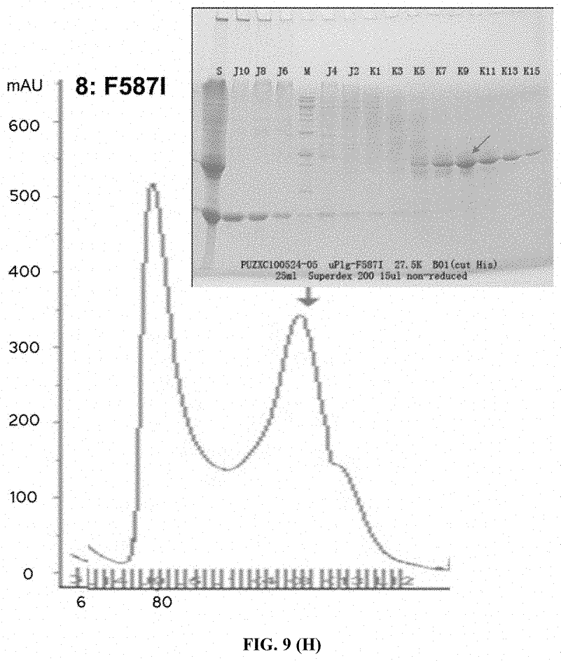

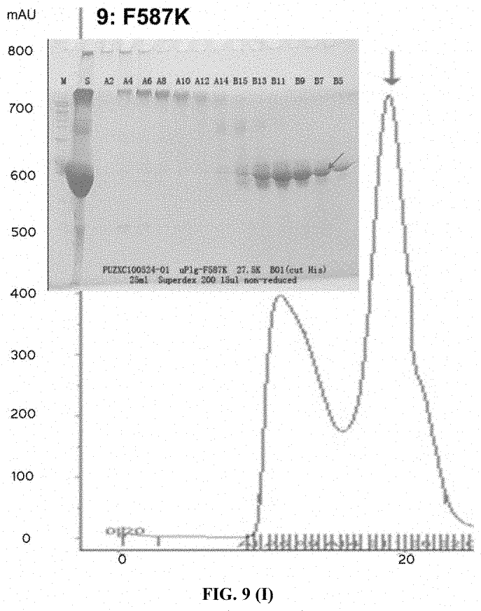

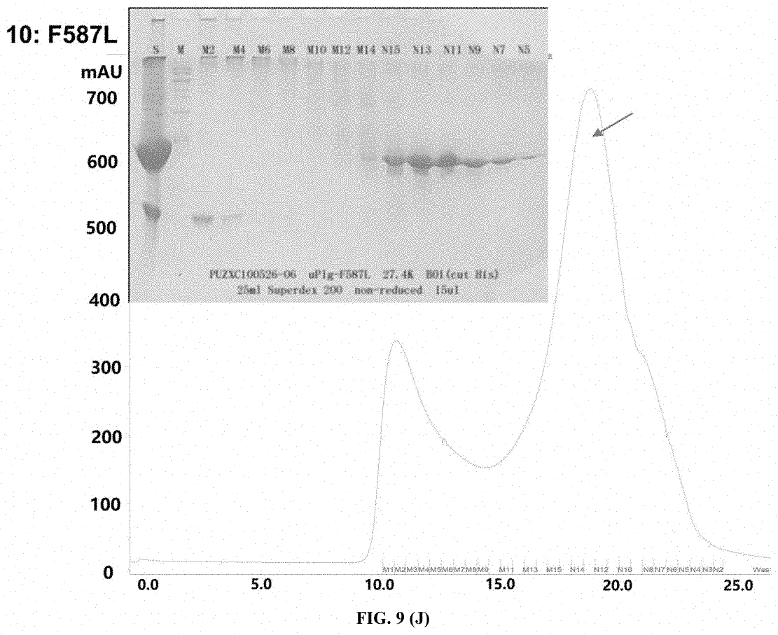

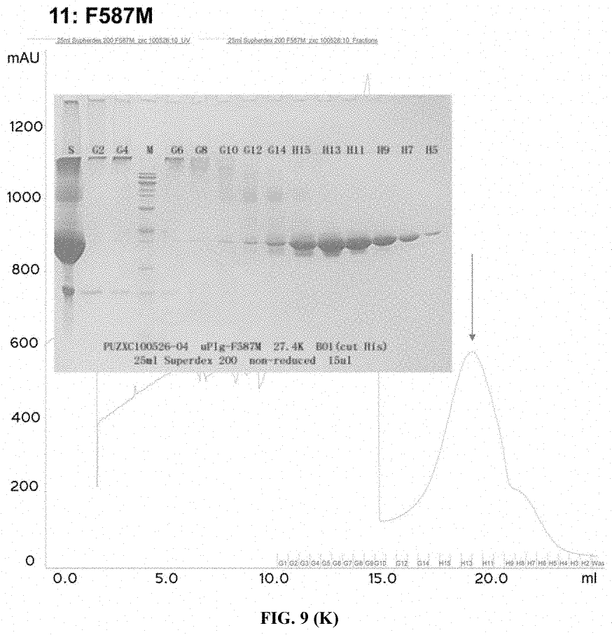

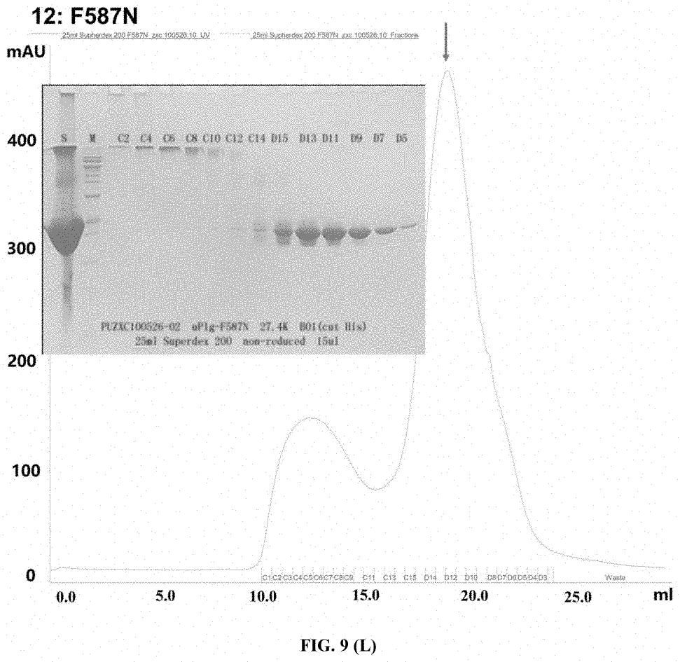

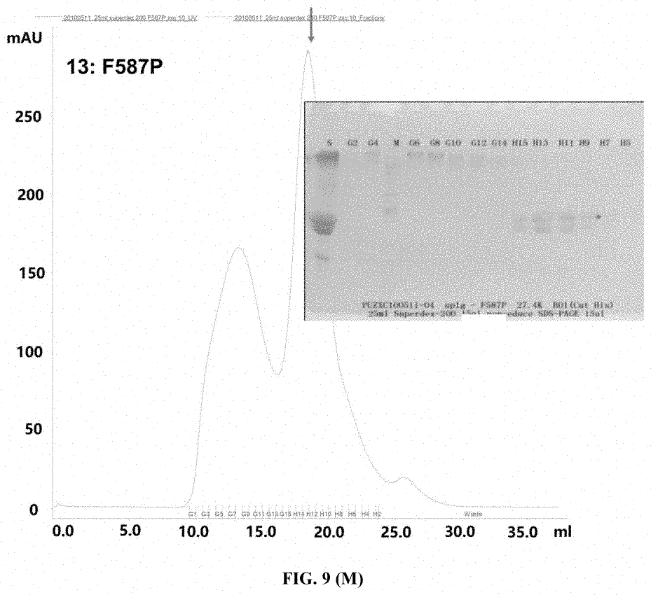

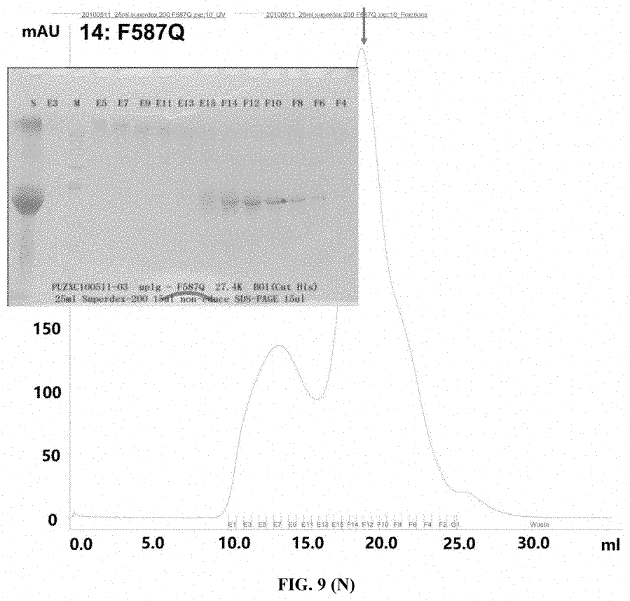

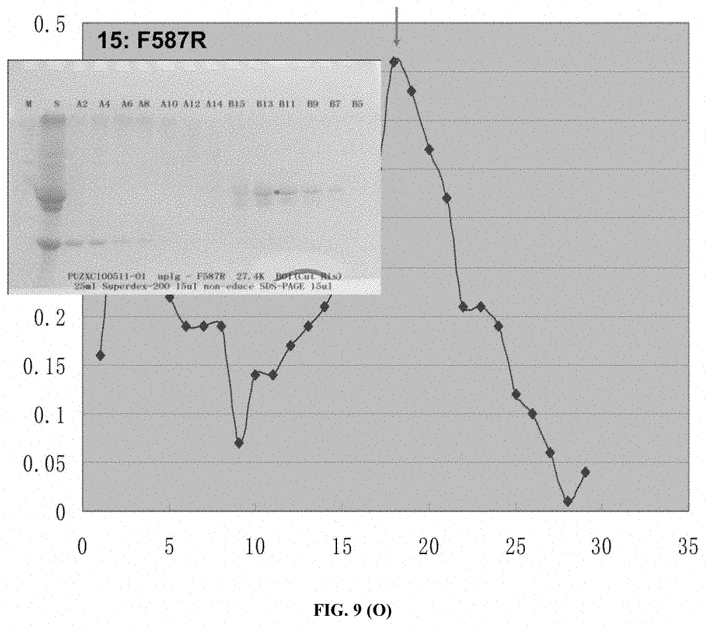

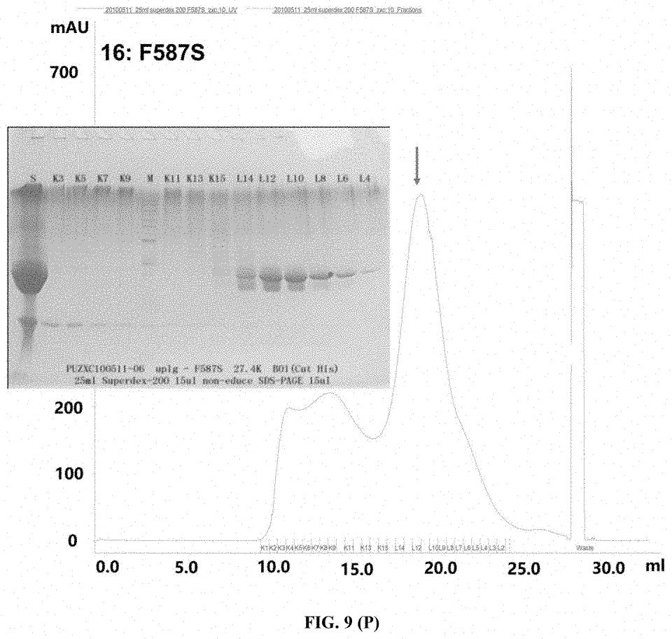

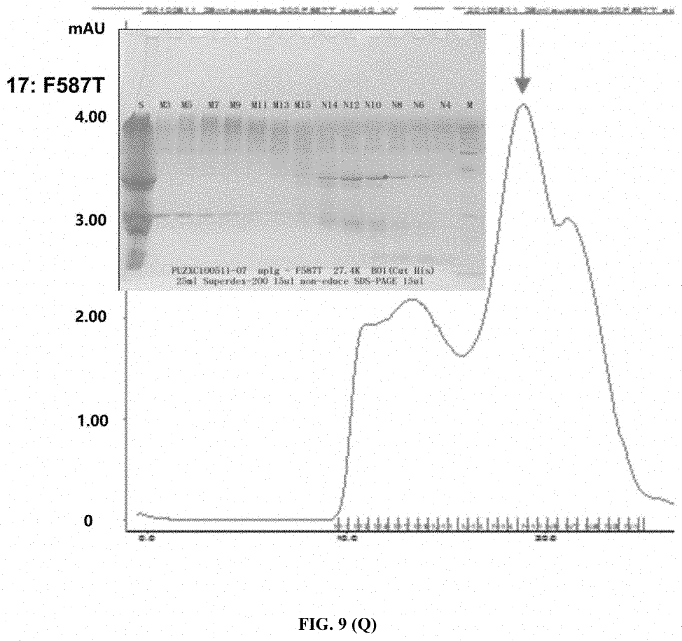

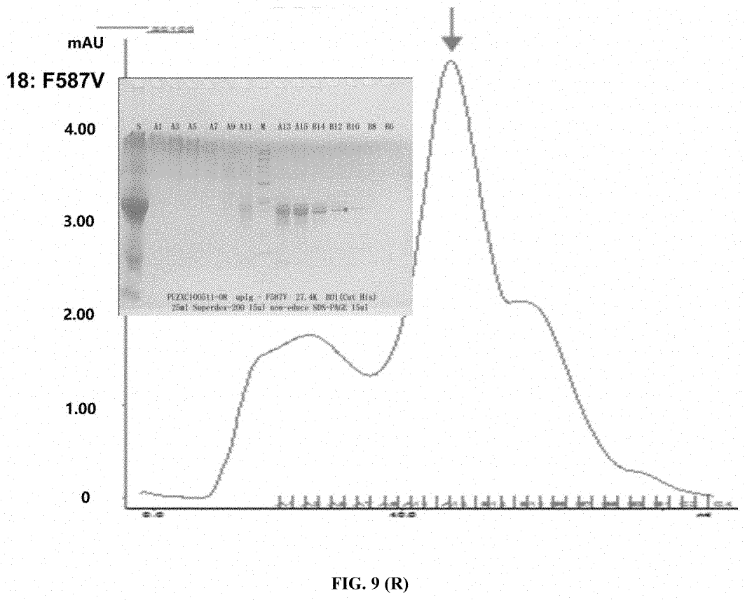

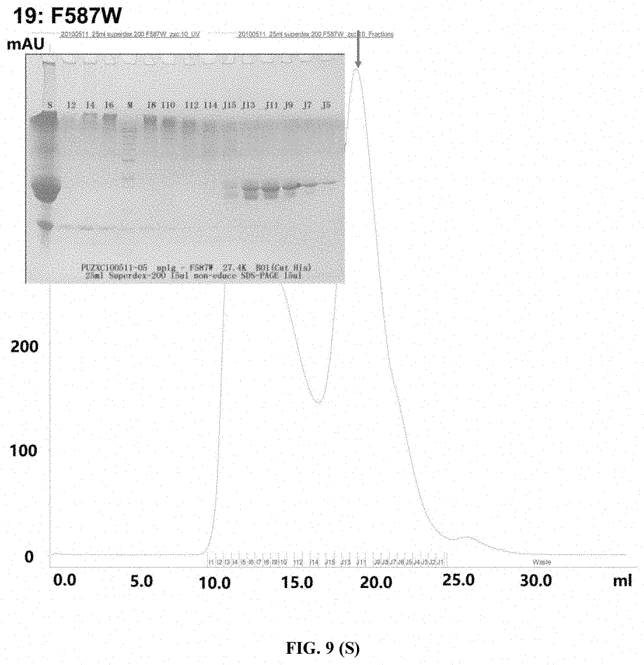

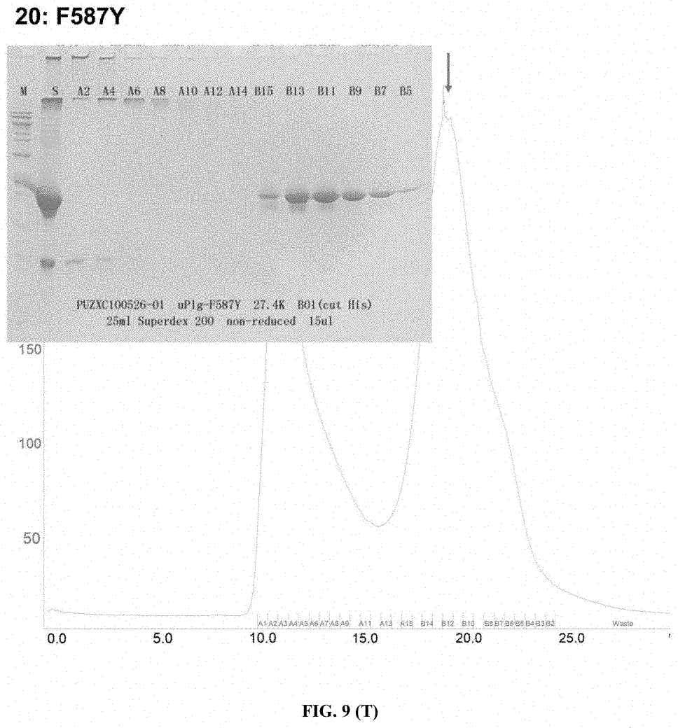

The instant disclosure also provides methods for saturation mutagenesis of the selected residues from the alanine scanning mutagenesis. Once a desired residue is identified from the alanine scanning mutagenesis, mutations to change to all other 18 amino acid residues (except wild type and alanine) will be made in the selected position, and further selection will be made to identify the best mutation in the selected position. Exemplified results of the first round of alanine scanning mutagenesis selection of the F587A mutant, and the second round of saturation mutagenesis at the F587 position are shown in FIGS. 8(A)-8(K), FIGS. 9(A)-9(T), Table 2, and Table 3.

The instant disclosure also provides methods for combinatorial mutagenesis of selected mutants. Desired mutants selected from the aforementioned alanine scanning mutagenesis and saturation mutagenesis may be combined in a single plasminogen polypeptide construct. The combined mutant may contain two mutations, three mutations, four mutations, and five or more mutations. “Optimized” mutant may be selected from the combinatorial mutagenesis strategy that is not only have good catalytic activity and resist to α2-AP inhibition, but also have enhanced activity toward certain peptide pathogens such as the β-Amyloid peptide and reduced activity toward other substrate such as fibrin.

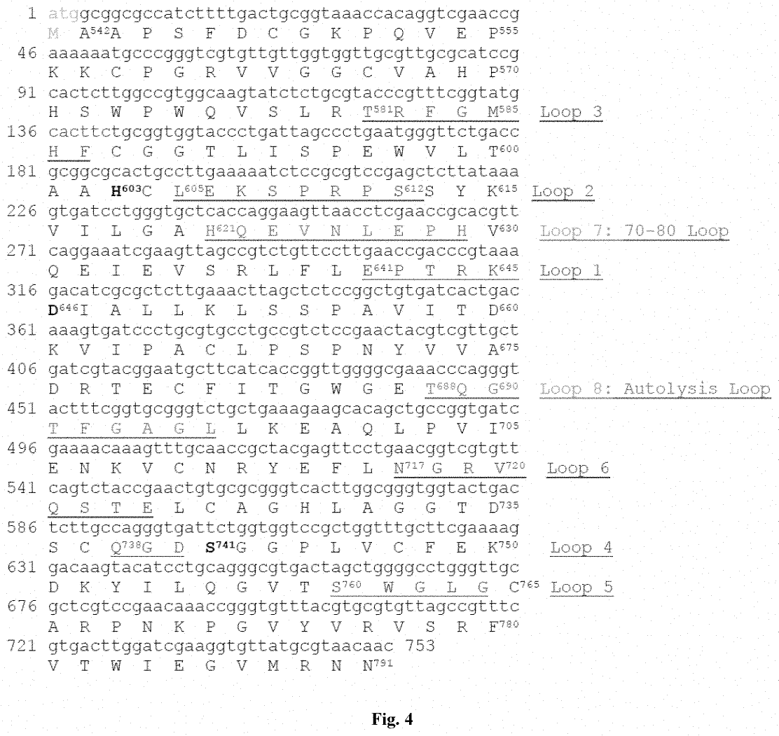

The instant disclosure also provides methods for mutagenesis design and method. FIG. 4 shows the amino acid sequence and the selected loop sequences of μPlg. The design is an illustration but not limitation for the present disclosure. Other amino acid residues may also be changed to achieve the same goal. A total of 54 amino acids are designed to be changed to alanine separately using Stratagene's QuickChange® Site-Directed Mutagenesis Kits with primer design method, or any other mutagenesis methods such as PCR-based mutagenesis known in the art. The primer design method may be used to design and synthesis each primer pair, and the kit may be used with wild-type expression plasmid as the template, to perform 54 mutagenesis reactions. Each isolated mutant expression plasmid will be sequence verified, and expressed in the same way as the wild-type.

The instant disclosure also provides methods for selection of the mutant variants. First, small scale inclusion body expression, purification and refolding screening may be performed. The sequence verified mutant plasmids may be transformed into a BL21(DE3) strain of bacteria for expression as described in reference Medynski, D.; Tuan, M.; Liu, W.; Wu, S.; Lin, X., Refolding, purification, and activation of miniplasminogen and microplasminogen isolated from E. coli inclusion bodies. Protein Expr Purif 2007, 52 (2), 395-402. Initially, about 200 ml culture of wild-type μPlg and each of the 49 mutants may be grown, yielding about 200 mg of highly purified inclusion bodies for each construct. The inclusion bodies may be dissolved in: 8 M urea, 20 mM Tris, 1 mM Glycine, 10 mM beta-mercaptoethanol, 10 mM DTT, 1 mM reduced glutathion (GSH), 0.1 mM oxidized glutathion (GSSG), pH 9.0, with OD₂₈₀ adjusted to about 2. A rapid dilution method may be used for small-scale refolding for each mutant. These techniques are published and are within the skill of art.

The instant disclosure also provides methods for selection of the mutant variants by any high-throughput selection methods known in the skill of art. For example, large-scale colony based or plate-based methods may be designed to select mutants with the desired properties.

The instant disclosure also provides methods for use 96-well based activity assay to screen mutants. The refolded samples may be directly used for proteolytic assay using 96-well plates. The basic assay procedure is as follows: To a 1.5 ml Eppendorf Tube, add 4 μl of the refolded zymogen (OD₂₈₀=0.5), 4 μl of urokinase (OD₂₈₀=0.02), and 392 μl of buffer containing 50 mM Tris, 38 mM NaCl, 0.001% Tween, pH 8.8. Urokinase is used to activate μPlg to μPlm. The reaction is incubated at 37° C. for 30 min. Next, to a Corning 96 well flat bottom, non-binding surface, polystyrene assay plate (3641), 100 μl activated enzyme may be aliquoted into 3 wells for each reaction (3 assay samples from each reaction). A multi-pipetor may then be used to add 50 μl of 1 mM S-2403 (Chromogenix) substrate into the wells to start the reaction. A₄₀₅ may be monitored immediately after the start of the reaction using a plate reader, and the initial slope represents the initial reaction rates. Mutants that have similar or higher biological activity than the wild-type may be selected for the next α2-AP inhibition assay.

The instant disclosure also provides methods for α2-AP inhibition assay and candidate identification. About seven different inhibition reactions may be performed for wild-type (control) and each of the selected mutants. The concentration of μPlg in the reaction is about 5 μM and in the initial activation mix is about 50 nM. After activation at 37° C. for 30 min, α2-AP (purified from human serum, SIGMA) may be added to a final concentration of 0, 10, 20, 30, 40, 50, and 100 nM. Inhibition kinetics may be calculated according to published procedures, for example, Wiman, B.; Boman, L.; Collen, D., On the kinetics of the reaction between human antiplasmin and a low-molecular-weight form of plasmin. Eur J Biochem 1978, 87 (1), 143-6, or Aoki, N.; Sumi, Y.; Miura, O.; Hirosawa, S., Human alpha 2-plasmin inhibitor. Methods Enymol 1993, 223, 185-97. FIG. 8(A)-8(K) shows some of the results of the inhibition assay. From these inhibition data, the methods may identify about 5-10 escape mutants. For the identified mutants, larger scale (1-2 L) refolding may be performed; mutant proteins may be purified according to published procedures. More detailed kinetic studies may be performed for the purified mutants and finally one may obtain some desired mutants.

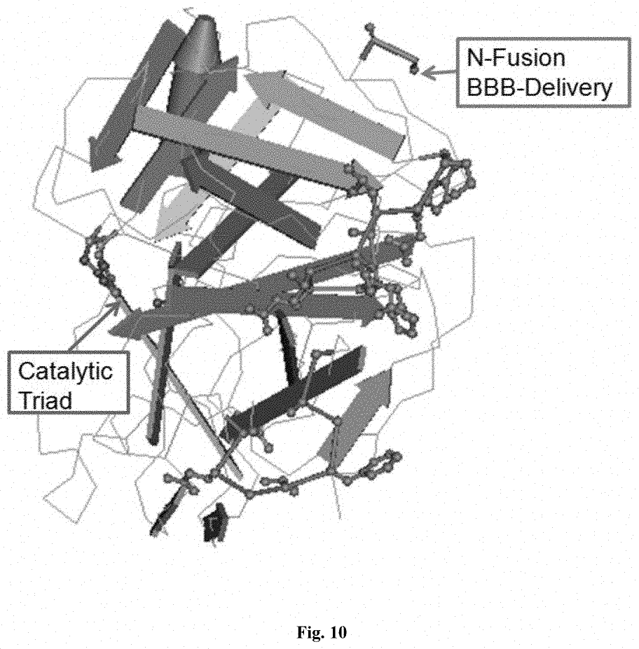

The instant disclosure also provides methods for fusion protein construction and selection for crossing the blood brain barrier (BBB). A “molecular Trojan horse” strategy is designed by engineering fusion partners to ferry the therapeutic plasminogen/plasmin polypeptides across the human BBB (FIG. 10). Fusion partners with proven receptor mediated BBB transcytosis properties are designed in order to select a high-efficient transporter for the selected μPlm construct, as shown in FIG. 8(A)-8(K). (1) Angiopep-2 (AP2) transports to the brain through LRP1 and has been successfully tested for brain delivery. AP2-μPlm (Bridge sequence: SGPGGGS): MTFFYGGSRGKRNNFKTEEY-SGPGGGS-μPlm (SEQ-ID NO:5). (2) The low-density lipoprotein receptor (LDLR)-binding domain of apolipoprotein B (ApoB) has proven transcytosis property across the BBB and has been successfully used in brain delivery of a therapeutic protein. ApoB-μPlm: MSSVIDALQYKLEGTT RLTRK RGLKLATALS LSNKFVEGSHNSTVSOPQQQS-μPlm (SEQ-ID NO:6).

The instant disclosure also provides methods for Cys-PEGylation scanning mutagenesis. As shown in FIG. 3(B), identified loops 7 and 8 (contact regions between μPlm and the γ-SK) as potential mutagenesis sites have been identified. It has been shown that γ-SK blocks α2-AP inhibition without interfering with μPlm activity, implying that the γ-SK-μPlm complex has the desired properties of being used as a possible drug candidate for AD. However, γ-SK is a bacterial protein and therefore immunogenicity may be an impassable hurdle for Aβ drug development. On the other hand, using polyethylene glycol (PEG) to “replace” the γ-SK domain in the complex may result in similar blockage of α2-AP inhibition and also retain native activity. PEGylated drugs are known to extend in vivo half life, reduce or eliminate immunogenicity, and have already been approved by the FDA. Two steps are designed for Cys-PEGylation screening at selected residues of loops 7 and 8 (FIG. 3(B)) and possibly other residues of loops 1-6 shown in FIG. 3(A). First, residues are identified according to preliminary alanine scanning mutagenesis results for Cys mutation, and the kinetic properties of the resulting Cys mutants will be characterized. Second, for Cys mutants that have desired kinetic properties, Cys-PEGylation will be performed and the PEGylated μPlm (PEG-μPlm) mutants will be characterized. Structurally (FIG. 3(B)), using polyethylene glycol to functionally “replace” the γ-domain of SK is a novel approach, and the implication of the resulting PEGylated μPlm can be far reaching in all aspects of drugable properties: native activity, blocking α2-AP inhibition, reducing or eliminating possible immunogenicity, and longer in vivo half-life.

The instant disclosure also provides methods for plasmin polypeptide mutant selection for further developing into pharmaceutical agent to treat diseases such as PAO, Alzheimer's disease, and lung fibrosis. The final mutant for the drug development may not be a mutant with a single amino acid change. With a more comprehensive combinatorial mutagenesis and saturation mutagenesis study, the disclosure methods may eventually select a mutant that may have amino acid changes at 1-10 or more positions of the plasmin polypeptide for a particular indication, and the eventual amino acid change may not be alanine. The initial alanine scanning is designed only for learning the sensitivity of enzymatic activity and α2-AP inhibition at each individual position. After this learning process, one will obtain the data for designing the next, more comprehensive study. The mutant selection criteria includes high activity (about 40-150% or higher wild-type activity), low sensitivity to α2-AP inhibition (about 0-60%/α2-AP inhibition assuming the wild-type inhibition is 100%), high refolding yield (comparable to or better than the wild-type refolding yield), and highly stable at certain formulation and conditions (comparable to the wild-type enzyme).

### Methods of Producing Plasminogen and Plasmin Polypeptides

The methods described below may be used to produce wild-type, mutant, fusion, and PEGylated variants of plasminogen polypeptides. The mutant construct variants are selected mutant constructs of plasminogen polypeptides that have desired pharmaceutical properties, including wild type like enzyme activity, resistance to α2-AP inhibition, ability to cross the BBB, and longer in vivo half-life and reduced immunogenicity in the PEGylated or other modified form.

The methods of the disclosure are typically practiced utilizing inclusion bodies containing plasminogen polypeptide or the designed mutants, such as plasminogen polypeptide produced in bacterial (e.g., E. coli) cells which have been engineered to produce the polypeptide and mutants, as the starting material, but any source of denatured or natively expressed plasminogen polypeptides and mutants may be used. The plasminogen may be from any species desired, and from any natural or non-natural plasminogen sequence, according to the practitioner's preference. The full length human plasminogen amino acid sequence including the signal sequence is shown in FIGS. 1(C1)-1(C3) and SEQ ID NO:1. Any polynucleotide sequences encoding the amino acid sequence may be used (such as changes which may improve expression in the host organism, i.e., “optimized” sequences, such as shown in FIG. 4 for μPlg).

Recombinant (e.g., bacterial, such as Escherichia coli, or yeast, such as Saccharomyces cerevisia or Pichia postoris, or insect, such as SF-9 cells, or mammalian, such as CHO cells) host cells may be engineered to produce plasminogen polypeptide using any convenient technology. Most commonly, a DNA sequence encoding the desired plasminogen polypeptide is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector.

The bacterial host cells are typically cultured in a liquid growth medium for production of plasminogen polypeptide under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

Other host cells, such as different yeast strains, may also be used in a liquid growth medium for production of plasminogen polypeptide under conditions appropriate to the host cells and expression vector. Preferably, the host cells are cultured in a yeast cell growth fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial or yeast host cells are cultured to the desired density (and after any necessary induction of expression), the cells are collected. Collection is typically conveniently effected by centrifugation of the growth medium, although any other convenient technique may be used. The collected host cells may be washed at this stage to remove traces of the growth medium, most typically by resuspension in a simple buffer followed by centrifugation (or other convenient cell collection method). At this point, the collected host cells (the “cell paste”) may be immediately processed in accordance with the disclosure, or it may be frozen for processing at a later time.

For yeast cell expression system, the cell paste are lysed with techniques within skill of the art, such as enzymatic digestion of the cell walls and sonication, and native plasminogen polypeptides or mutant variants of plasminogen peptides may be purified according to procedures described in previous literatures, such as described by Nagai et al (Recombinant human microplasmin: production and potential therapeutic properties. J Thromb Haemost 2003, 1 (2), 307-13.), or described for refolded proteins below.

For bacteria expression system, the bacterial cells of the cell paste are lysed to release the polypeptide-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0.1-0.2 M (it is apparently undesirable to use essentially zero ionic strength). Any suitable salt, including NaCl may be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g., centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100®), then recollecting the inclusion bodies. The washed inclusion bodies are then dissolved in a solubilization buffer.

The solubilization buffer comprises a high concentration of a chaotroph, one or more reducing agents, and a buffer that buffers the solution to a pH of about 8.0 to about 11.0. The solubilization buffer may optionally contain additional agents, such as redox reagents, cation chelating agents and scavengers to neutralize protein-damaging free-radicals.

The instant disclosure utilizes urea as an exemplary chaotroph in the refolding buffer, although guanidine hydrochloride (guanidine HCl) may also be used. Useful concentrations of urea in the solubilization buffer include about 4 M to about 8 M, about 5 M to about 8 M, about 6 M to about 8 M, about 7 M to about 8 M. When the chaotroph is guanidine HCl, useful concentrations include about 1 M to about 8 M, or about 4 M to about 6 M, or about 6 M.

The pH of the refolding buffer is high, viz., in excess of pH 7.0, for example 10.0. For example, the pH may be of about 8.0 to about 11.0, about 9.0 to about 11.0, or about 9.0 to 10.0. As will be apparent to those of skill in the art, any pH buffering agent (or combination of agents) which effectively buffer at these pH ranges are useful, although pH buffers which can buffer in the range between pH 8.0 to pH 11.0 are particularly useful. Useful pH buffering agents include tris (tris(hydroxymethyl)aminomethane), bicine (N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), AMPD (2-Amino-2-methyl-1,3-propanediol).N-(2-Hydroxyethyl)piperazine-N′-(4-butanesulfonic acid)), and the like. The pH buffering agent is added to a concentration that provides effective pH buffering, such as from about 10 mM to about 400 mM, about 75 mM to about 300 mM, about 200 mM, or about 20 mM.

Reducing agents are included in the solubilization buffer to reduce disulfide bonds and maintain cysteine residues in their reduced form. Useful reducing agents include β-mercaptoethanol, dithiothreitol, and the like. More than one reducing agents, such as both β-mercaptoethanol and dithiothreitol, may be used together. Additionally, the refolding buffer may contain disulfide reshuffling or “redox” reagents (e.g., a combination of oxidized and reduced glutathione). When the redox reagents are oxidized and reduced glutathione (GSSG and GSH, respectively), useful concentrations include about 0.1 mM to about 10 mM and useful ratios include but not limited to about 10:1, about 5:1, and about 1:1 (GSH:GSSG).

The solubilization buffer may contain additional components. For example, the solubilization buffer may contain a cation chelator such as a divalent cation chelator like ethylenediaminetetraacetic acid (EDTA) or ethylene glycol-bis(2-aminoethylether)-N,N,N′,N′-tetraacetic acid (EGTA). EDTA or EGTA is added to the solubilization buffer at a concentration of about 0.5 to about 5 mM, and commonly at about 1 mM. Additionally, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

An exemplary solubilization buffer comprises about the following concentrations of the following components: 8 M urea, 100 mM Tris, 1 mM glycine, 1 mM EDTA, 10 mM beta-mercaptoethanol, 10 mM dithiothreitol (DTT), 1 mM reduced glutathion (GSH), 0.1 mM oxidized glutathion (GSSG), pH 10.0.

The inclusion body/solubilization buffer mixture is incubated to allow full solubilization. The incubation period is generally from about 6 hours to about 24 hours, and more commonly about eight to about 8 hours or about 16 hours, or about 12 hours. Stirring may be applied, for example at a speed of 500 rpm. The inclusion body/solubilization buffer mixture incubation may be carried out at room temperature or at reduced temperature. For example, temperature may be between about 4° C. to about 10° C.

After the incubation is complete, the inclusion body/solubilization buffer mixture is clarified to remove undissolved inclusion bodies. Clarification of the mixture may be accomplished by any convenient means, such as filtration (e.g., by using 0.2-5.0 μm filter) or by centrifugation, or both. Clarification should be carried out at reduced temperature, such as at about 4° to about 10° C.

The clarified mixture is then diluted using the same solubilization buffer to achieve the appropriate protein concentration for refolding. Protein concentration may be determined using any convenient technique, such as Bradford assay, light absorption at 280 nm (A₂₈₀), and the like. A solution having from about 0.5 mg/ml to about 10 mg/ml (e.g., about 2 mg/ml) is appropriate for use in the instant methods. If desired, this mixture may be held, refrigerated (e.g. at 4° C.), for later processing, although the mixture is not normally held for more than about four weeks.

The concentration-adjusted inclusion body solution is first rapidly diluted about 20 fold with a refolding buffer. The dilution is performed by adding inclusion body solution into the refolding buffer. The inclusion body solution may be diluted about 5 to about 100 fold, about 10 to about 50 fold, about 10 to about 25 fold, about 15 to about 25 fold with the refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml. The concentration of urea or guanidine HCl in the diluted inclusion body solution may be about 1 M to about 3 M for urea; and about 0.5 M to about 2 M for guanidine HCl. This concentration of urea or guanidine HCl may be achieved by just diluting the inclusion body/solubilization buffer, or added into the refolding buffer.

The refolding buffer contains a pH buffer and L-arginine. The refolding buffer may also contain a low concentration of chaotroph, a disulfide reshuffling reagent, and a divalent cation chelator. These reagents may be added to the refolding buffer. The refolding buffer may include additional agents, such as free-radical scavengers. “Rapid” dilution, within the context of the disclosure means over a period of less than about 25 minutes, and the dilution process is generally carried out during periods of about one minute to about 25 minutes, or about five to about 20 minutes. The diluted solubilized plasminogen polypeptide solution is typically held for one to two hours following the completion of the rapid dilution process.

The refolding buffer may contain about 0.05 M to about 1.0 M arginine. The refolding buffer may also contain about 0.05 M to about 2.0 M urea. The refolding buffer may also contain glycerol (e.g., about 5 to about 20%) or sucrose (about 5 to about 30%).

The pH of the refolding buffer may be the same as or different from the solubilization buffer. The pH of the refolding buffer may be from about 8.0 to about 10.0, from about 8.5 to about 9.5, or from about 9.0 to about 9.5. In some embodiments, the refolding buffer has a pH of about 9.0 (e.g., for refolding mPlg and mutant variants). In some embodiments, the refolding buffer has a pH of about 9.5 (e.g., for refolding μPlg and mutant variants). The pH buffering agent in the refolding buffer may be any buffering agent or combination of buffering agents that are effective pH buffers at pH levels of about 8 to about 9 or about 10 or about 10.5. Useful pH buffering agents include tris (tris(hydroxymethyl)aminomethane), bicine (N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), and AMPD (2-Amino-2-methyl-1,3-propanediol).N-(2-Hydroxyethyl)piperazine-N′-(4-butanesulfonic acid)). The pH buffering agent is added to a concentration that provides effective pH buffering, such as from about 10 to about 150 mM, about 50 to about 150 mM, about 75 mM to about 125 mM, or about 100 mM.

The redox reagents included in the refolding buffer must be effective in ‘shuffling’ cysteine sulfhydryl groups between their oxidized and reduced states. The redox environment of the refolding reaction may be adjusted by manipulating the concentration of the redox reagents. When the redox reagents are oxidized and reduced glutathione (GSSG and GSH, respectively), the inventor has found that useful concentrations include about 0.005 mM to about 0.05 mM, about 0.1 mM to about 11 mM and useful ratios include about 10:1, about 5:1, and about 1:1 (GSH:GSSG).

The divalent cation chelator may be any molecule that effectively chelates Ca⁺⁺ and other divalent cations. Exemplary cation chelators for use in the refolding buffer include ethylenediaminetetraacetic acid (EDTA) or ethylene glycol-bis(2-aminoethylether)-N,N,N′,N′-tetraacetic acid (EGTA). When EDTA or EDTA is the divalent cation chelator, it is added to the refolding buffer at a concentration of about 0.5 to about 5 mM, and commonly at about 1 mM.

Additional components useful in the refolding buffer include free-radical scavengers. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM).

The refolding buffer may further comprise a protease inhibitor, such as Phenylmethylsulfonyl fluoride (PMSF), to prevent autoactivation and non-specific cleavage.

An exemplary refolding buffer comprises Tris and arginine at pH about 9.0 or about 9.5. In some embodiments, the refolding buffer comprises about 20 mM Tris and about 0.2 M arginine at pH about 9.5. In some embodiments, the refolding buffer comprises about 20 mM Tris, about 0.2 M arginine, about 0.05 mM PMSF, at pH about 9.0.

The refolding reaction is incubated for a period of about 1 to 2 hours to about 18 to 24 hours. The refolding reaction may be carried out at room temperature (e.g., about 18-20° C.) or at slightly reduced temperature (e.g., about 14-16° C.), depending on the preferences of the practitioner and the availability facilities.

Following the refolding, properly refolded plasminogen polypeptide may be concentrated, buffer exchanged, and further purified. Concentration/buffer exchange of the refolded protein may be accomplished using any convenient technique, such as ultrafiltration, diafilitration, chromatography (e.g., ion-exchange, hydrophobic interaction, or affinity chromatography) and the like. Where practical, it is preferred that concentration be carried out at reduced temperature (e.g., about 4-10° C.).

While any convenient protein purification protocol may be used. In some embodiments, three types of chromatography may be used for purification. For example, size exclusion chromatography (SEC), ion exchange chromatography (IEC), and affinity chromatography may be used.

Size exclusion chromatography (SEC) may be performed using any convenient chromatography medium which separates properly folded plasminogen polypeptide from unfolded and multimeric form. The inventor has found that media having the ability to size fractionate proteins of about 104 to about 6×10⁵ daltons (globular proteins) are useful for this step. Exemplary SEC media include Sephacryl®300, Sephacryl® 200, Sephacryl® 100, Superdex™ 200, and Superdex™ 75. This step may also be used to perform buffer exchange, if so desired. The exact conditions for SEC will depend on the exact chromatography media selected, whether buffer exchange is to be accomplished, the requirements of any later purification steps, and other factors known to those of skill in the art.

The properly folded plasminogen polypeptide may be further purified utilizing ion exchange chromatography, for example, cation exchange or anion exchange chromatography. Cation exchange chromatography (i.e., SP-Sepharose from GE Healthcare) may be washed with a buffer containing 20 mM phosphate, 10% sucrose, pH 6.5, with 0-1 M NaCl gradient.

The properly folded plasminogen polypeptide may be further purified utilizing affinity chromatography, for example, benzamidine affinity column (e.g., obtained from GE Healthcare).

Refolded plasminogen polypeptide may be treated with plasminogen activator, such as urokinase, tissue plasminogen activator (tPA), streptokinase, staphylokinase, to generate biologically active plasmin, which may be further purified using any of the methods described above for plasminogen or methods known in the art.

Biological activity of plasmin polypeptide produced from the properly folded recombinant plasminogen polypeptide produced in accordance with the disclosure may be measured using any acceptable assay method known in the art. An exemplary method of measuring plasmin activity is described herein in Examples, which measures amidolysis of plasmin chromogenic substrate S-2403 (L-Pyroglutamyl-L-Phenylalanyl-L-Lysine-para-Nitroaniline hydrochloride).

Biological activity of plasmin polypeptide produced from the properly folded recombinant mutant variants of plasminogen polypeptide produced in accordance with the disclosure may also be measured using fibrin as substrate. The wild-type plasmin polypeptide may be used as a positive control in measuring the cleavage of fibrin substrate. The cleavage may be measured using methods known in the art.

Biological activity of plasmin polypeptide produced from the properly folded recombinant mutant variants of plasminogen polypeptide produced in accordance with the disclosure may also be measured using a β-amyloid peptide as substrate. The wild-type plasmin polypeptide may be used as a positive control, and the cleaved β-amyloid peptide may be measured by any methods known in the art, such as HPLC, MS, and LC/MS.

The disclosure also provides an aqueous composition comprising a plasminogen or plasmin polypeptide mutants produced by the method described herein. In some embodiments, the composition may further comprise a pharmaceutically acceptable excipient. Pharmaceutically acceptable excipients are known in the art.

Purified mutant plasmin polypeptide may be stored in low pH buffer with sucrose. For example, citric acid at pH of less than about 5.0 or less than about 4.0, or about 3.1 may be used. An exemplary buffer comprises about 20 mM citric acid, about 7.5% sucrose, about 0.15 M NaCl, at pH about 3.1.

Purified Cys-mutants of plasminogen polypeptide constructs may be PEGylated at the installed Cys-residue. Detailed procedure is described below.

As is well understood in the art, all concentrations and pH values need not be exact and reference to a given value reflects standard usage in the art, does not mean that the value cannot vary.

The following examples provide a detailed description of the production of properly folded recombinant plasminogen and plasmin polypeptides (wild-type or mutant) in accordance with the methods of the disclosure and the characterization thereof. These examples are not intended to limit the disclosure in any way.

### Examples

### Materials and Methods:

Some of the procedures have been published. These procedures represent essential techniques to support the present disclosure.

Construction of μPlg expression vectors: Synthetic cDNAs encoding μPlg (sequence shown in FIG. 4) optimized for expression in E. coli were synthesized. The protein sequences (sequence shown in FIG. 4 and SEQ ID NO:3) used to generate the cDNAs were obtained from the protein sequence for full length human plasminogen (Accession #P00747) from the ExPASY server (www.expasy.org) of the Swiss Institute of Bioinformatics. The optimized cDNA encoding μPlg was inserted into the unique BamHI/Xho1 sites of a custom pET11a expression vector with a modified expanded multiple cloning site, creating the plasmid pET11a-μPlg.

Expression, Refolding, and Purification using μPlg as an example. The μPlg construct was expressed in E. coli BL21 (DE3) in a shaker flask system and expressed exclusively as inclusion bodies. E. coli BL21 (DE3) cells were transformed with pET11a-μPlg plasmid and plated onto PA-0.5G/Ampicillin plates and incubated for 16 hours at 37° C. One colony was used to inoculate 50 ml PA-0.5G liquid media and incubated in a shaking flask at 37° C. for 16 hours to an OD₆₀₀ of 0.5-1.6 and immediately stored at 4° C. to create a working stock. The working stock (0.5 ml) is used to inoculate 500 ml ZYP-5052 liquid media and incubated 16 hours at 37° C., 300 RPM to an OD₆w of 8-10 in a 2.8 L Fernbach flask. For larger scale production, 6-12 L was produced in multiple 2.8 L Fernbach shaker flasks. Alternatively, The μPlg construct was expressed in E. coli BL21(DE3) in a shaker flask system using an auto-induction procedure developed by StudierF. W., Protein production by auto-induction in high density shaking cultures. Protein Expr Purif 2005, 41 (1), 207-34), with higher production yield.

Isolation and Solubilization of Inclusion Bodies: After induction, the cells were harvested and centrifuged at 7,000 RPM, 4° C. for 10 minutes. The pellet was then resuspended in 50 ml of 50 mM Tris, 25% sucrose, 1 mM EDTA, 10 mM DTT, pH 8.0. Lysozyme (100 mg) was added and the solution was stirred for 30 minutes at room temperature. A buffer (125 ml) containing 20 mM Tris, pH 7.5, 1% NaDeoxycholate, 1% Triton X-100, 10 mM NaCl, 10 mM DTT was added and the solution was stirred for an additional 30 minutes and then frozen at −85° C. for 20 hours. The solution was then thawed in a 37° C. water bath for 3 hours and homogenized using a Branson ultrasonic horn at 70% amplitude, 30 second pulse, 10 second pause for 6 cycles. The solution was brought to 1 L with 50 mM Tris, 100 mM NaCl, 0.5% Triton X-100, 1 mM EDTA, 1 mM DTT, pH 8.0 (Triton Solution) and stirred at 800 RPM, 4° C. for 1 hour. The inclusion bodies were then centrifuged at 7,000 RPM, 4° C. for 1 hour, and the resulting pellet was resuspended with 1 L of Triton Solution and stirred again at 800 RPM, 4° C. for 1 hour. The cycle of centrifugation and resuspension in Triton Solution was repeated 3 more times followed by 4 washes with 50 mM Tris, 1 mM EDTA, 1 mM DTT (Tris Solution). After the last wash with Tris Solution, the pellet was solubilized in 10 ml 8M Urea, 100 mM Tris, 1 mM Glycine, 100 mM Beta-ME, pH 10.5 and stirred for 16 hours at 300 RPM, 4° C. The solubilized inclusion bodies were then ultracentrifuged at 30,000 RPM, 4° C. for 30 minutes and characterized by A280 and non-reducing SDS-PAGE.

Refolding of denatured μPlg: The purified inclusion body preps were diluted in a 8 M urea buffer (8 M urea, 0.1 M Tris, 1 mM glycine, 1 mM EDTA, 10 mM R-ME, 10 mM dithiothreitol (DTT), 1 mM reduced glutathione (GSH), 0.1 mM oxidized glutathione (GSSG), pH 10 with a final concentration of 2 mg/ml as an initial step just before rapid dilution refolding was initiated. The inclusion body was incubated from 10 min to about 2 hours. Subsequently this solution was rapidly diluted into 20 volumes 20 mM Tris, 0.2 M arginine, pH 9.0. The final volume in the experiments ranged from 4 L to 16 L. The solutions were allowed to refold over night at 18° C. (approximately 16 hrs) and then were transferred to a 4° C. cold room for an additional 48 hrs. The refolded protein is then concentrated by N₂ ultrafiltration, and purified by various types of column chromatography.

Structural and physical tests to determine refolding success. Even before the protein is tested for activity, there are several structural and physical criteria which give a good indication that the protein is correctly folded: (i) No apparent precipitation during refolding process. (ii) Proteins concentrated to high concentrations by ultrafiltration stay in solution. (iii) Gel filtration produces a “folded” peak at the appropriate size for the particular protein. Partially or totally unfolded proteins and multimers elute with the void volume and are easily separated away. (iv) Non-reduced SDS-PAGE identifies purified protein with right molecular weight. Furthermore, the protein should give a sharp, rather than fuzzy, band on the gel, indicating that there are not multiple folded forms but a unique one. Of course, these convenient tests do not substitute for sophisticated methods of structural analysis such as dynamic light scattering (DLS), circular dichroism.

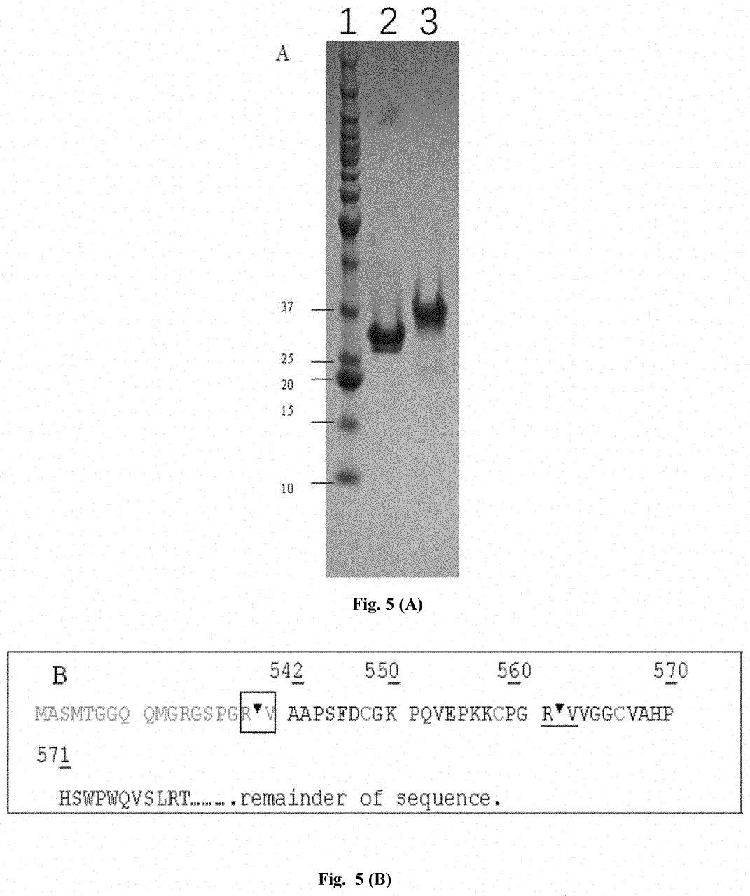

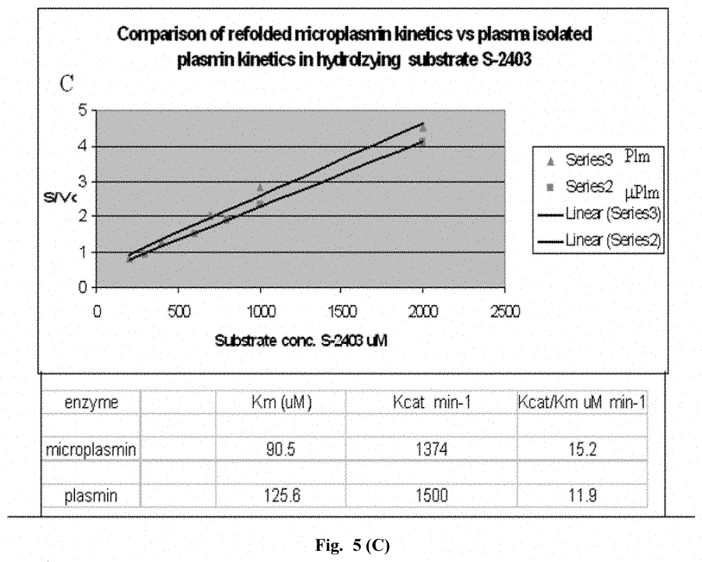

Activation of μPlg→μPlm using uPA. used uPA (urokinase) to activate μPlg (FIG. 5(A), 5(B)) have been use This material was then used to characterize the kinetic parameters of μPlm in hydrolyzing the synthetic amidolytic substrate S-2403. FIG. 5(A) shows μPlg before activation with uPA under non-reducing and reducing conditions (lanes 2 and 3 respectively). The non-reduced material was treated with uPA at 37° C. for 45 minutes at a uPA:μPlg ratio of 1:20, then run on SDS-PAGE and blotted onto an Immobilon-P® PVDF transfer membrane (Millipore, Bedford, Mass.) blotting paper; the urokinase digest products (μPlm) were NH₂ sequenced by Edman degradation. The cleavage pattern of the urokinase gave the 2 anticipated cleavage sites (R▾VA⁵⁴² and R^{561▾}V⁵⁶²) (FIG. 5(B)).

Kinetic comparison of μPlm with commercially available plasmin. Kinetic characterization of the μPlm was compared with commercially available plasmin (Sigma, Cat #P1867). A fixed concentration of either μPlm or plasmin (155 nM) was mixed with a range of concentrations of S-2403 (200-2000 μM) in quadruplicate using a titertek micropipetter in a 96-well microplate. The A_{405 nm} was measured at 30 second intervals for 5-15 minutes using a Spectramax 384 plate reader (Molecular Devices, Sunnyvale, Calif.). The raw data was converted to μM amounts and analyzed using a Hanes plot. As indicated in FIG. 5(C) the μPlm and plasmin had fairly similar Kₘ's and K_{cat}s; overall, the catalytic efficiency of μPlm was 1.27× higher than plasmin (15.2 μM min⁻¹ vs. 11.9 μM min⁻¹).

Inhibition of μPm and mutants by α₂-AP. The inhibition of μPlm and Mutants by α₂-AP (purified from human serum, Abcam, Cambrige, Md.) was investigated at 37° C., in 50 mM Tris-HCl, 100 mM NaCl buffer, pH 7.4. IC₅₀ values for inhibition were determined at substrate concentration of 400 μM, 100 nM μPlg, and various concentrations of α₂-AP ranging from 4 nM to 200 nM. The α₂-AP was mixed with the substrate, and the reaction was initiated by the addition of 100 μL substrate with α₂-AP to 10 μL μPlm. The progress curves were monitored for 30 min using a Spectramax 96 plate reader. The remaining activity of μPlm in the presence of α₂-AP were compared with the activity in the absence of the inhibitor, and IC₅₀ values were calculated by fitting the data using a non-linear regression with the GraFit software according to published procedures, for example, Wiman, B.; Boman, L.; Collen, D., On the kinetics of the reaction between human antiplasmin and a low-molecular-weight form of plasmin. Eur J Biochem 1978, 87 (1), 143-6, or Aoki, N.; Sumi, Y.; Miura, O.; Hirosawa, S., Human alpha 2-plasmin inhibitor. Methods Enzymol 1993, 223, 185-97. Some of the results are shown in FIGS. 8(A)-8(K) and Table 3.

Cys-PEGylation. The refolded and purified protein in 20 mM Tris, 200 mM NaCl, 1 mM DTT, pH 7.6 buffer will be exchanged over a PD-10 (BioRad) column pre-equilibrated with 50 mM NaPi pH 7.5, 200 mM NaCl to remove DTT and change the pH to 7.5 according to the manufacturer's protocol. The buffer-exchange process is usually performed twice to be absolutely certain there were no trace levels of DTT present because this reducing agent interferes with the pegylation reaction. The buffer-exchanged protein is quantitated by molar extinction. Solid mPEG-20 (polyethylene glycol maleimide 20 having approximate molecular weight of 22 KDa, Nektar, Huntsville, Ala.) stored at −20° C. under argon gas is added to the solution of the mutant plasminogen polypeptide construct at a molar ratio of about 5:1 to about 10:1 and incubated at 37° C. for 60-120 minutes. The reaction will be stopped by adding 20 mM DTT and incubating for an additional 5 minutes at 37° C. The reaction mixture is then run over SEC or ion exchange columns to separate pegylated from unpegylated protein. Pegylation will be verified by SDS-PAGE and MALDI-TOF mass spectrometry.

### Results:

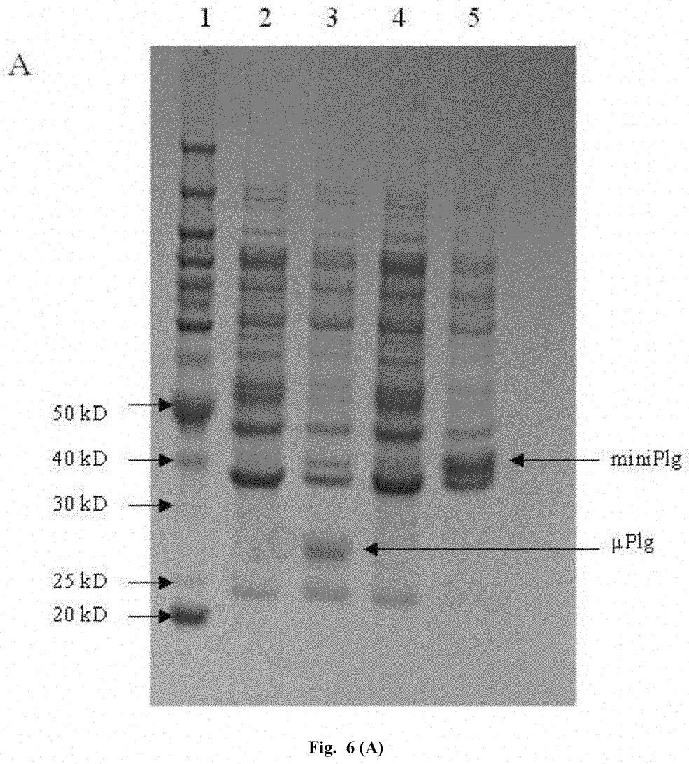

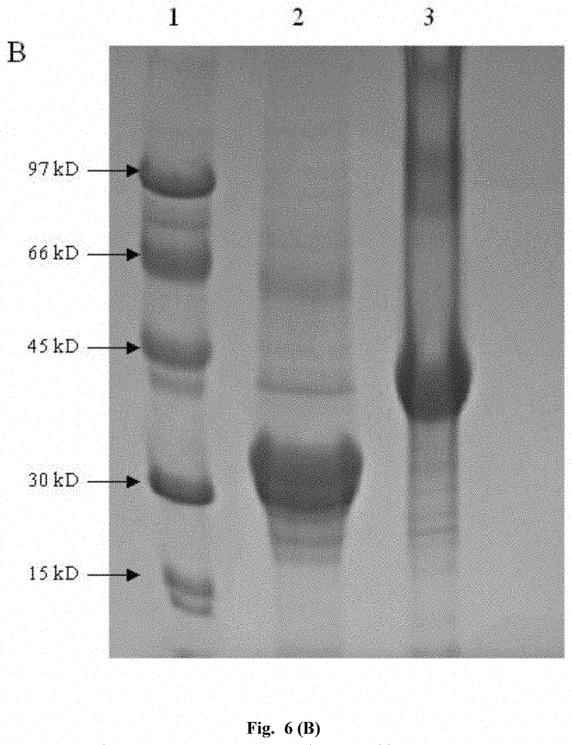

High level expression of μPlg and mPIg as inclusion bodies: FIG. 6(A) depicts specific expression of μPlg and mPlg expression in the presence and absence of IPTG. FIG. 6(B) depicts purified inclusion bodies for μPlg and mPlg. The proteins in the inclusion bodies are estimated to be at least 90% pure before refolding is initiated. Although the data shown in FIG. 6(A) involved growth in LB broth and induction with IPTG, routinely the auto-induction system of Studier and fermentor fermentation for large scale preparation of inclusion bodies are utilized, since a 5-50 fold higher yields of inclusion bodies can be obtained.

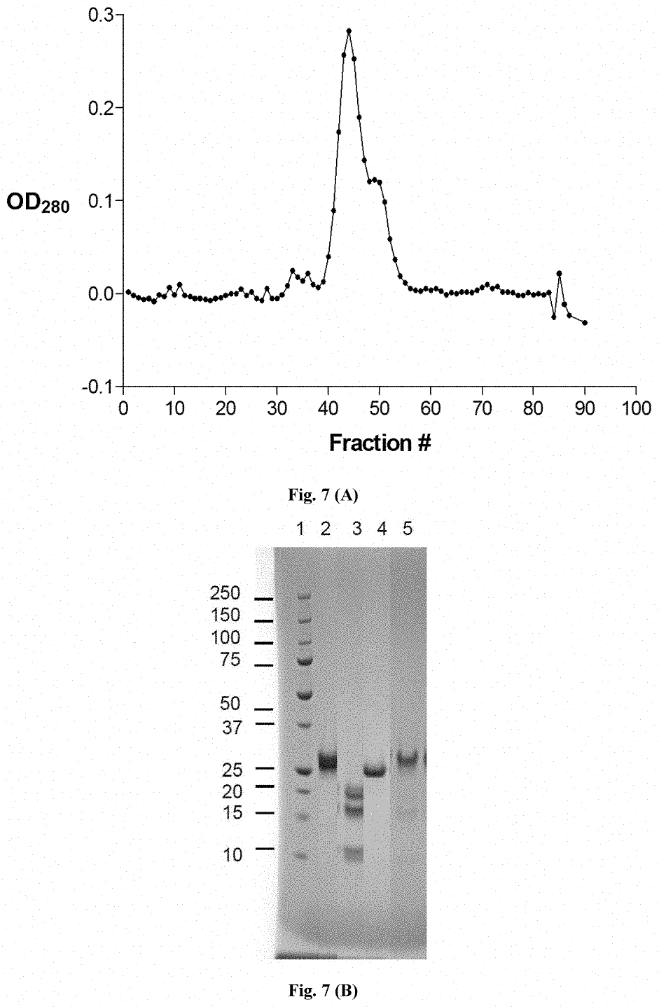

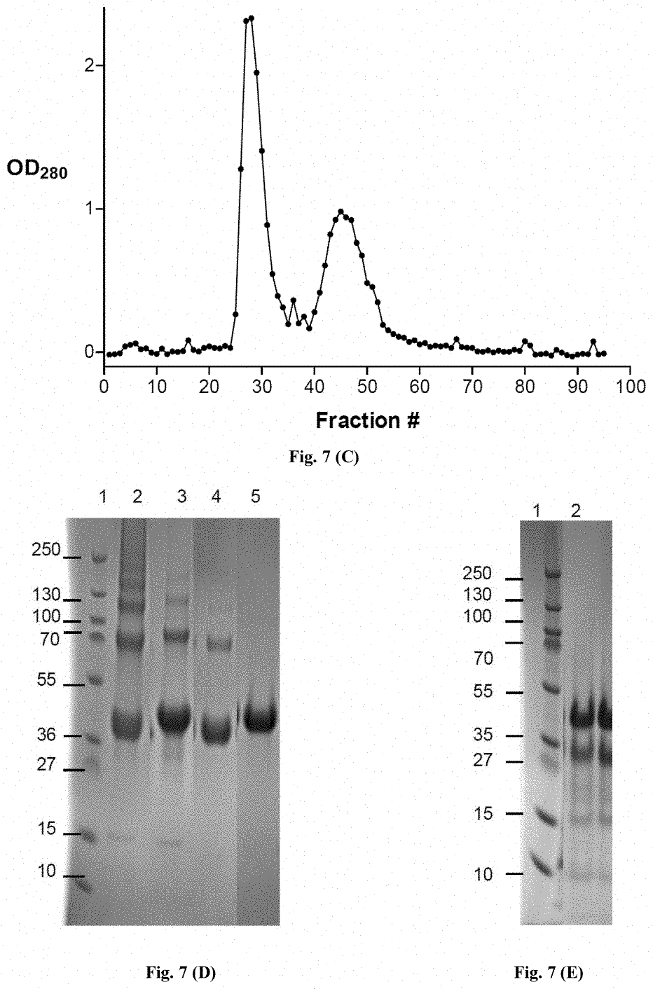

Refolding and Purification of μPlg and mPIg: μPlg has six disulfide bridges and mPlg has nine disulfide bridges. Therefore, it is not surprising that there would be a significant amount of incorrectly formed disulfides and multimeric forms of the protein generated during the refolding process. Before arriving at final refolding conditions, a small scale “screening refolding” process consisting of a matrix in which more than 40 refolding conditions were examined. Criterion for selection in the initial screening included lack of visible protein precipitate during refolding or in subsequent concentration steps for SEC, and elution of protein peaks from the SEC with a mobility identical to refolded monomeric forms of the protein. SEC is used as an important tool in post-refolding purification because multimeric, unfolded, soluble forms of the protein can be easily separated from refolded monomer. Ultimately, the refolding conditions summarized in Table 1 were selected because they generated the highest percent of monomeric form of μPlg or mPlg in the SEC chromatographic step. They also presented a favorable activity profile when the proteins were activated to either μPlm or mPlm and examined in a functional assay. Representative chromatograms of isolated μPlg and mPlg are presented in FIGS. 7A and 7C, respectively. Overall, approximately 20-40% of total protein refolded from inclusion bodies, estimated to be >90% pure at the outset, was isolated in the final monomeric refolded form at >98% purity (Table 1, FIGS. 7B, 7D).

Kinetic Characterization of μPlm and mPlm: Refolded μPlm and mPlm were compared kinetically with commercially available plasmin at 21° C. using plasmin chromogenic substrate S-2403. As described in detail above, a fixed concentration of μPlm, mPlm, or plasmin was mixed with a range of concentrations of S-2403 (300-3000 μM). FIG. 5(C) summarized a comparison between plasmin and μPlm. The kinetic parameters for the two molecules were relatively similar. These initial data established that the active site conformation of the serine protease domain of the three molecules is very similar, validated the refolding methodology.

Alanine scanning mutagenesis. To test the alanine scanning mutagenesis strategy, 54 mutant μPlg expression vectors have been constructed. The mutants are described in FIGS. 3A-3B and FIG. 4. As an example, the results of testing the expression are presented, refolding, and purification of 10 mutants, along with the wild-type (WT) μPlg enzyme. The expressed mutants belong to the loop 2 (K607A, S608A, P609a, P611A), loop3 (T581A, R582A, G584A, M585A, F587A), and 70-80 loop (Q622A) shown in FIGS. 3(A), 3(B) and FIG. 4. FIGS. 8(A)-8(K) show some of the results, which are discussed briefly below. (1) The figures show that the WT and mutant μPlm can be purified with a one-step SEC purification after refolding, exemplified in FIGS. 8(A)-8(C). This simplified purification procedure allows for the proposed high-throughput screening of μPlm mutants. (2) Kinetic data (exemplified in FIG. 8(D) and summarized in FIGS. 8(E), 8(F)) shows that, out of 10 mutants tested, six (T581A, R582A, M585A, F587A, K607A, S608A) have higher specific activity than the WT enzyme, suggesting that selecting mutants with similar and higher activities than the WT enzyme is achievable. (3) It is known that α2-AP reacts with μPlm to form an enzymatically inactive stoichiometric 1:1 complex, with an initially fast second order reversible reaction followed by a slower first order irreversible reaction. At equal molar or higher concentrations, α2-AP completely inhibits the activity of the WT μPlm, with a typical “bi-phases” kinetic process shown in FIG. 8(G) and FIG. 8(J). The inhibition kinetics of the mutant F587A, however, is not “typical”. FIG. 8(H) shows that even at 2 times concentration of α2-AP and long term incubation, there is still about 50% activity remaining for the mutant F587A. FIG. 8(F) and FIG. 8(I) show that through the exemplified testing of the 10 mutants, a F587A mutant have been identified, which not only has higher specific activity than the WT enzyme, but also can partially escape α2-AP inhibition. FIG. 3(A) shows the structure and sequence of the loop-3, and FIG. 8(K) shows a space filling model of the loop-3 and the active site triad residues. It is apparent from the figure that the aromatic ring of the F587 is located near the active site S741, and exposed to the solvent. This residue seems to be one of the contact residues to provide binding energies when α2-AP “approaching” and trying to bind to μPlm, and the mutation of the F587 weakened both the kinetics and thermodynamics of the binding interactions.

Saturation mutagenesis at the 587 position. Mutagenesis method was used to change amino acid residue at the F587 position to all other natural amino acid. All of the 19 mutant expression vectors were prepared and sequence verified. FIGS. 9(A)-9(T) shows the SEC purification profile of all of the F587 mutants, and Table 2 shows that the refolding yields, calculated based on the results of FIGS. 9(A)-9(T), of most of the mutants are either similar or better than the WT.

Kinetic and inhibition studies of F587 mutants. As shown in Table 3, the kinetic efficiencies (Kcal/Kₘ) of the μPm WT and all nineteen mutants at the 587 position were measured. 8 mutants that have similar or better Kcal/Kₘ value than the WT were selected, and the α2-AP inhibition which is expressed as IC₅₀ were measured. The results show that all the tested mutants have higher IC₅₀, or more resisting α2-AP inhibition, than the WT. These results clearly validated the design and applicability of the present disclosure.

TABLE 1
Summary of Refolding Protocol for μPlasmin and Miniplasmin

| Protein | μPlasmin | miniPlasmin |
|---|---|---|
| Refolding | 20 mM Tris, | 20 mM Tris, |
| Buffer | 0.2M Arginine | 0.2M Arginine |
| | 0.4M urea, | 0.02 mM PMSF |
| | 0.05 mM GSH, | 0.4M urea, |
| | 0.005 mM GSSG, | 0.05 mM GSH, |
| | 0.5 mM β-ME, | 0.005 mM GSSG, |
| | 0.05 mM EDTA | 0.5 mM β-ME, |
| | pH 9.5 | 0.05 mM EDTA |
| | | pH 9.5 |
| Refolding | 0.1 mg/mL | 0.1 mg/mL |
| Concentration | | |
| Refolding | Overnight | Overnight |
| Time | at 18° C., | at 18° C., |
| | then 48 hrs | then 48 hrs |
| | at 4° C. | at 4° C. |
| Total Protein | 20-25% after | 15-20% after |
| Monomer | SP IEX | Superdex 75 SEC |
| Yield After 1^{st} | chromatography | |
| Step of | | |
| Purification | | |
| Total Protein | 10% after | 10% after |
| Monomer | Superdex 75 SEC | Superdex 200 SEC |
| Yield After 2^{nd} | | |
| Step of | | |
| Purification | | |

TABLE 2
Summary of the Refolding Yield of F587 Mutants

| # | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| aa | WT | A | C | D | E | G | H | I | K | L | M | N | P | Q | R | S | T | V | W | Y |
| % | 40 | 51 | 64 | 87 | 62 | 50 | 93 | 55 | 62 | 77 | 47 | 76 | 59 | 74 | 42 | 58 | 69 | 77 | 51 | 61 |

TABLE 3
Kinetic parameters of the WT μPlm and 19 mutants at the 587 position

| | | | | | | Kcat/Km | IC50 |
|---|---|---|---|---|---|---|---|
| | | Kcat | Km | Kcat/Km | IC50 | Fold | Fold |
| | μPlg | (min⁻¹) | (μM) | (μM⁻¹ mm⁻¹) | (nM) | Increase | Increase |
| 1 | WT | 131.0 ± 4.1 | 211.5 ± 19.1 | 0.6 | 37.9 ± 2.7 | 1.0 | 1.0 |
| 2 | F587A | 516.4 ± 14.6 | 299.1 ± 22.2 | 1.7 | 149.9 ± 10.8 | 2.8 | 4.0 |
| 3 | F587C | 92.1 ± 2.2 | 167.3 ± 12.3 | 0.6 | | 0.9 | |
| 4 | F587D | 300.2 ± 8.1 | 444.4 ± 25.4 | 0.7 | 106.0 ± 12.0 | 1.1 | 2.8 |
| 5 | F587E | 133.4 ± 2.7 | 429.0 ± 19.6 | 0.3 | | 0.5 | |
| 6 | F587G | 113.2 ± 6.5 | 333.4 ± 43.3 | 0.3 | | 0.5 | |
| 7 | F587H | 83.3 ± 1.9 | 296.1 ± 17.3 | 0.3 | | 0.5 | |
| 8 | F587I | 263.7 ± 9.7 | 243.9 ± 24.9 | 1.1 | 46.4 ± 5.2 | 1.7 | 1.2 |
| 9 | F587K | 85.7 ± 2.3 | 217.7 ± 16.4 | 0.4 | | 0.6 | |
| 10 | F587L | 123.4 ± 3.6 | 240.4 ± 17.7 | 0.5 | | 0.8 | |
| 11 | F587M | 212.6 ± 5.1 | 258.2 ± 15.6 | 0.8 | 77.6 ± 6.8 | 1.3 | 2.0 |
| 12 | F587N | 215.5 ± 6.1 | 356.5 ± 23.1 | 0.6 | 104.7 ± 10.3 | 1.0 | 2.8 |
| 13 | F587P | | | | | No Activity | |
| 14 | F587Q | 392.0 ± 14.9 | 258.9 ± 25.3 | 1.5 | 63.2 ± 5.1 | 2.4 | 1.7 |
| 15 | F587R | 211.9 ± 8.5 | 266.2 ± 26.1 | 0.8 | 163.8 ± 18.2 | 1.3 | 4.3 |
| 16 | F587S | 88.5 ± 5.3 | 380.8 ± 57.3 | 0.2 | | 0.4 | |
| 17 | F587T | 51.3 ± 1.8 | 249.9 ± 25.7 | 0.2 | | 0.3 | |
| 18 | F587V | 271.7 ± 3.9 | 302.4 ± 11.5 | 0.9 | 65.2 ± 5.0 | 1.5 | 1.7 |
| 19 | F587W | 145.4 ± 5.4 | 452.5 ± 37.8 | 0.3 | | 0.5 | |
| 20 | F587Y | 164.0 ± 6.5 | 385.7 ± 34.7 | 0.4 | | 0.7 | |

What is claimed is:

1. A method for treating and/or preventing diseases in patient caused by a pathogenic polypeptide, comprising:
administrating to the patient in need an effective dose of a mutant and/or fusion plasminogen or plasmin polypeptide therapeutics in a pharmaceutical formulation.

2. The method of claim 1, wherein the diseases are thromboembolism related diseases including peripheral arterial occlusion, deep vein thrombosis, acute pulmonary embolism, and acute ischemic stroke.

3. The method of claim 1, wherein the diseases are amyloid peptide caused diseases including Alzheimer's disease caused by a β-Amyloid peptide.

4. The method of claim 1, wherein the diseases are caused by human tissue damages resulting from various causes including pulmonary fibrosis.

5. The method of claim 1, wherein the diseases are caused by decreased in vivo plasminogen and plasmin polypeptide content or activity; wherein the diseases includes disseminate intravascular coagulation, sepsis, leukemia, hyaline membrane disease, cardiovascular diseases, Alzheimer's disease, pulmonary fibrosis, and liver diseases.

6. The method of claim 1, wherein the mutant and/or fusion plasminogen and plasmin polypeptide is selected by the following methods:
(a) performing alanine scanning mutagenesis of the surface residues of the catalytic domain of plasmin (μPlasmin) that is involved in complex formation with α2-Antiplasmin; and selecting mutants that have similar catalytic activity as that of the wild type enzyme but also resist to α2-Antiplasmin inhibition; wherein the alanine scanning mutagenesis of changing all residues of μPlasmin individually into alanine and selecting mutants that have similar catalytic activity as that of the wild type enzyme but also resist to α2-Antiplasmin inhibition is used;
(b) performing saturation mutagenesis for the mutations selected from the alanine scanning mutagenesis of method (a); and selecting mutants that have similar catalytic activity as that of the wild type enzyme but also resist to α2-Antiplasmin inhibition;
(c) performing combinatorial mutagenesis of constructing combined mutations from mutants selected using method (a) or (b); and selecting mutants that have optimal catalytic activity toward a particular polypeptide pathogen but also resist to α2-Antiplasmin inhibition;
(d) performing Cys-PEGylation scanning mutagenesis of the surface residues of the catalytic domain of plasmin (μPlasmin) that is involved in complex formation with α2-Antiplasmin; and selecting mutants that have similar catalytic activity as that of the wild type enzyme but also resist to α2-Antiplasmin inhibition; wherein, the Cys-PEGylation scanning mutagenesis of changing all residues of μPlasmin individually into PEGylated-Cysteine and selecting mutants that have similar catalytic activity as that of the wild type enzyme but also resist to α2-Antiplasmin inhibition is used;
(e) performing albumination through albumin-fusion or fatty acid modification of plasmin (μPlasmin) residues that is involved in complex formation with α2-Antiplasmin; and selecting constructs that have similar catalytic activity as that of the wild type enzyme but also resist to α2-Antiplasmin inhibition;
(f) performing modification of a mutant plasmin (μPlasmin) that resulting in similar catalytic activity as that of the wild type enzyme but also resist to α2-Antiplasmin inhibition, including a better in vivo half-life and substrate specificity; wherein the substrate specificity comprises reduced catalytic efficiency toward fibrin or better catalytic efficiency toward small peptide substrates including β-amyloid peptides; wherein the modification includes PEGylation and albumination; and
(g) performing fusion construct of modified mutant plasmin (μPlasmin) that resulting in similar catalytic activity as that of the wild type enzyme but also resist to α2-Antiplasmin inhibition, having a better in vivo half-life, and is able to cross the blood brain barrier (BBB), for treating CNS diseases including Alzheimer's Disease.

7. The method of claim 1, wherein the mutant plasmin polypeptide is derived from the corresponding mutant plasminogen polypeptide by treating with a plasminogen activator.

8. The method of claim 7, wherein the mutant plasmin polypeptide is selected to be biologically active in cleaving and detoxifying pathogenic polypeptides and is also resisting α2-antiplasmin inhibition.

9. The method of claim 8, wherein the mutant recombinant plasmin polypeptide contains >80% of the amino acid residues 542-791 of the SEQ ID NO. 1.

10. The method of claim 8, wherein the mutant recombinant plasmin polypeptide contains >80% of the amino acid residues 440-791 of the SEQ ID NO. 1.

11. The method of claim 8, wherein the mutant recombinant plasmin polypeptide contains >80% of the amino acid residues 1-791 of the SEQ ID NO. 1.

12. The method of claim 11, wherein one or more kringle structures or other non-catalytic structures are deleted.

13. The method of claim 1, wherein the mutant plasminogen polypeptide is expressed and purified from an E. coli expression system.

14. The method of claim 1, wherein the mutant recombinant plasminogen polypeptide is expressed and purified from a yeast expression system.

15. The method of claim 1, wherein the mutant recombinant plasminogen polypeptide is expressed and purified from an insect or a mammalian expression system.

16. A composition comprising a mutant plasminogen and plasmin polypeptide produced by the method of claim 8.

17. The composition of claim 16, further comprising a pharmaceutically acceptable excipient.

18. The composition of claim 17, further comprising the delivery of the mutant plasminogen and plasmin polypeptide in a pharmaceutically acceptable excipient through intravenous, subcutaneous, submuscular, and aerosol routes; wherein the aerosol delivery comprises using inhale devices including Nebulizers, metered dose inhalers, and dry powder inhalers.


SEQUENCE LISTING (6 sequences)

SEQ ID NO: 1 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 2 - protein, 352 aa, Homo sapiens
ASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - protein, 250 aa, Homo sapiens
AAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 4 - protein, 491 aa, Homo sapiens
MALLWGLLVLSWSCLQGPCSVFSPVSAMEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 5 - protein, 19 aa, Homo sapiens
TFFYGGSRGKRNNFKTEEY

SEQ ID NO: 6 - protein, 44 aa, Homo sapiens
SSVIDALQYKLEGTTRLTRKRGLKLATALSLSNKFVEGSHNSTV